(19) United States
(12) Patent Application Publication
(10) Pub. No.: US 2002/0147168 A1
(43) Pub. Date: Oct. 10, 2002

(54) Single-step excision means

(76) Inventors: Brian Peter Surin, Rivett (AU); Robert Charles De Feyter, Monash (AU); Michael Wayne Graham, St. Lucia (AU); Peter Michael Waterhouse, O' Connor (AU); Paul Konrad Keese, Curtin (AU); Shahjahan Ali, Ngunnawal (AU)

Correspondence Address: GREENLEE WINNER AND SULLIVAN P C, 5370 MANHATTAN CIRCLE, SUITE 201, BOULDER, CO, 80303, US

(21) Appl. No.: 09/850,846

(22) Filed: May 7, 2001

(30) Foreign Application Priority Data
Mar. 29, 1996 (AU) PN 9031

(51) Int. Cl. A61K 48/00; C12N 15/87

(52) U.S. Cl. 514/44; 435/320.1; 435/455

(57) ABSTRACT

The present invention is directed to the genetic transformation using multiple genetic sequences, wherein one of said genetic sequences encodes a polypeptide possessing excision activity, specifically a site-specific recombinase activity linked to a transgene unit and the use of this genetic construct in the removal of transgenes therefrom. The present invention provides the means to produce genetically-transformed organisms, in particular plants, in which selectable marker genes have been removed, thereby facilitating multiple sequential genetic transformation events using the same selectable marker gene. Accordingly, the invention provides the means for regulating transgene expression in genetically-manipulated organisms, for example to promote differentiation, de-differentiation, or any unidirectional developmental shift of a target cell which requires the time-specific expression of a particular gene. The invention is particularly suited to the promotion of specific organogeneses in plants using organogenesis-promoting transgenes, wherein the organs which subsequently develop in said plants are genetically transformed with a desired gene but lack organogenesis-promoting transgenes.

The present invention relates generally to genetic sequences and their use in the production of genetically-transformed organisms. More particularly, the present invention is directed to the genetic transformation using multiple genetic sequences, wherein one of said genetic sequences encodes a polypeptide possessing excision activity, specifically a site-specific recombinase activity, and uses of same in the removal of transgenes therefrom. The present invention provides the means to selectively remove transgenes from genetically-transformed organisms. The present invention provides the means to produce genetically-transformed organisms, in particular plants, in which selectable marker genes have been removed, thereby facilitating multiple sequential genetic transformation events using the same selectable marker gene. Additionally, the present invention may be used to transiently integrate any genetic material into the chromosome of an organism, such that it may be expressed only while so integrated. Accordingly, this aspect of the invention provides the means for tightly regulating transgene expression in genetically-manipulated organisms, for example to promote differentiation, de-differentiation, or any unidirectional developmental shift of a target cell which requires the time-specific expression of a particular gene. The invention is particularly suited to the promotion of specific organogeneses in plants using organogenesis-promoting transgenes, wherein the organs which subsequently develop in said plants are genetically transformed with a desired gene but lack organogenesis-promoting transgenes.

Bibliographic details of the publications referred to in this specification by author are collected at the end of the description.

Throughout this specification, unless the context requires otherwise, the word “comprise” or variations such as “comprises” or “comprising” will be understood to imply the inclusion of a stated element or integer or group of elements or integers, but not the exclusion of any other element or integer or group of elements or integers.

Improvements in recombinant DNA technology have produced dramatic changes to the nature of the pharmaceutical and agricultural industries. In particular, methods for the introduction of desirable genetic traits into a wide range of organisms have led to the production of transgenic organisms which are of significant economic value. For example, transgenic crop plants have been produced with improved disease resistance to a range of plant pathogens and insect pests, digestibility and shelf-life, higher productivity and producing novel secondary metabolites.

Known procedures for the production of transgenic organisms mostly involve the introduction thereto of one or more reporter genes and/or selectable marker genes encoding herbicide or antibiotic resistance to facilitate the detection and/or selection of cells which express the gene, however much concern has been raised about the escape of such genes into the environment. Such concerns are of particular significance to transgenic plants which are capable of reproducing asexually or which comprises a significantly out-breeding population pollinated by wind or insects. Clearly, the removal of selectable marker genes from transgenic organisms prior to their release would alleviate such concerns. In the case of reporter genes, their continued expression in a transgenic organism may represent a biological load which compromises productivity gains.

Furthermore, the expression of certain transgenes such as selectable marker genes and reporter genes is often only desirable or necessary during the initial stages of transformation, in order to assess the efficiency of transformation and to identify and/or select transformed cells. Continued expression of such genes in transformed, regenerated tissues may constitute a genetic load on the organism thus obtained. As a consequence, it is often desirable to remove reporter genes from transgenic material prior to commercial application.

Furthermore, as each transformation event requires some form of selection, the introduction of multiple novel traits into an organism is limited by the availability of different selectable marker genes. The removal of selectable marker genes following each transformation event would permit the introduction of multiple genes in stages, using the same selectable marker gene.

Those skilled in the art are also aware that not all selectable marker genes are of equal utility in the genetic transformation of a particular organism. Clearly, the removal of marker genes following transformation would enable the re-use of an optimum selection system.

Known systems for the removal of selected genes from transgenic cells involve the use of site-specific recombination systems, for example the cre/lox system (Dale and Ow, 1991; Russell et al, 1992) and the flp/frt system (Lloyd and Davis, 1994; Lyznik et al, 1905) which comprise a loci for DNA recombination flanking a selected gene, specifically lox or frt genetic sequences, combination with a recombinase, cre or flp, which specifically contacts said loci, producing site-specific recombination and deletion of the selected gene. In particular, European Patent No. 0228009 (E. I. Du Pont de Nemours and Company) published Apr. 29, 1987 discloses a method for producing site-specific recombination of DNA in yeast utilising the cre/lox system, wherein yeast is transformed with a first DNA sequence comprising a regulatory nucleotide sequence and a cre gene and a second DNA sequence comprising a pre-selected DNA segment flanked by two lox sites such that, upon activation of the regulatory nucleotide sequence, expression of the cre gene is effected thereby producing site-specific recombination of DNA and deletion of the pre-selected DNA segment. U.S. Pat. No. 4,959,317 (E. I. Du Pont de Nemours and Company) filed Apr. 29, 1987 and International Patent Application No. PCT/US90107295 (E. I. Du Pont de Nemours and Company) filed Dec. 19, 1990 also disclose the use of the cre/lox system in eukaryotic cells.

Furthermore, International Patent Application No. PCT/US92/05640 (The United States of America as represented by the Secretary of Agriculture, USA) filed Jul. 6, 1992 discloses a method of excising and segregating selectable marker genes in higher plants using site-specific recombination systems such as the cre/lox or flp/frt systems wherein plant cells are first transformed with a recombinant vector which contains a plant-expressible selectable marker gene operably linked to loci for DNA recombination and the selectable marker gene is subsequently excised from transformed plants by further transforming the plant cells with a second recombinant vector which contains a plant-expressible, site-specific recombinase gene or, alternatively, by cross-pollinating the first-mentioned transformed plant with a second transformed plant which expresses a recombinant site-specific recombinase. As a consequence, the selectable marker gene contained in the first-mentioned transformed plant is excised. According to PCT/US92/05640, the recombinant site-specific recombinase gene is also linked to a selectable marker gene which must be removed to produce a plant which is free of selectable marker transgenes. This approach, therefore, requires at least one generation of conventional plant breeding to remove the second selectable marker gene.

A requirement for the operation of site-specific recombination systems is that the loci for DNA recombination and the recombinase enzyme contact each other in vivo, which means that they must both be present in the same cell. The prior art means for excising unwanted transgenes from genetically-transformed cells all involve either multiple transformation events or sexual crossing to produce a single cell comprising both the loci for DNA recombination and the site-specific recombinase.

Where multiple transformations are performed to achieve this end, several selectable marker genes must also be employed, thereby making their removal from the transformed plant material more difficult. As International Patent Application No. PCT/US92/05640 (USDA) demonstrates, the removal of unwanted selectable marker genes following multiple transformation events, requires a resort to conventional breeding approaches. These approaches thus involve extensive manipulation of transgenic material.

Furthermore, since all of the prior art requires some degree of breeding, the approaches taken are not generally applicable to asexually propagating species or clonally-propagated genetic stocks.

In work leading up to the present invention, the inventors sought to develop an improved system for the removal, deletion or excision of transgenes from genetically-transformed cells, which overcomes the disadvantages of the prior art. Accordingly, the inventors have produced a genetic construct which facilitates the precise excision of genetic material in a single generation, without the need for sexual crossing. The inventors have further defined an efficient method for the single-step removal, deletion or excision of transgenes, in particular selectable marker genes, reporter genes, hormone-biosynthesis genes, hormone-encoding genes or genetic sequences which encode one or more polypeptides capable of regulating hormone levels, from transformed cells.

Accordingly, one aspect of the present invention provides a genetic construct comprising a first expression cassette which contains a recombinase genetic unit linked to a transgene unit, wherein said expression cassette is flanked by two recombination loci placed upstream and downstream thereof.

The present invention is particularly useful in the removal, deletion or excision of transgenes from vegetatively- or clonally propagated species such as, but not limited to, potatoes, sweet potatoes, Jerusalem artichoke, taro or yams, fibre or wood tree crops such as Eucalyptus ssp. or Pinus ssp., aspen, ornamental plants such as roses, fuschias, azaleas carnations, camelias or gardenias, citrus crops such as oranges, lemons, grapefruit, tangerines or limes, fruit tress such as apples or pears, berry fruits such as strawberry, raspberry, loganberry or blackberry, tropical crops such as sugarcane, tobacco, bananas, plantain or pineapples or asparagus, amongst others.

The invention also permits the introduction of several unlinked transgenes into a single cell via independent transformation events, using the same selectable marker gene or reporter gene.

Reference herein to a “gene” or “genes” is to be taken in its broadest context and includes:

(i) a classical genomic gene consisting of transcriptional and/or translational regulatory sequences and/or a coding region and/or non-translated sequences (i.e. introns, 5′- and 3′-untranslated sequences); and/or

(ii) mRNA or cDNA corresponding to the coding regions (i.e. exons) and 5′- and 3′-untranslated sequences of the gene: and/or

(iii) a structural region corresponding to the coding regions (i.e. exons) optionally further comprising untranslated sequences and/or a heterologous promoter sequence which consists of transcriptional and/or translational regulatory regions capable of conferring expression characteristics on said structural region.

The term “gene” is also used to describe synthetic or fusion molecules encoding all or part of a functional product.

As used herein, the term “transgene” shall be taken to refer to any nucleic acid molecule, including, but not limited to DNA, cDNA, mRNA, tRNA, rRNA, synthetic oligonucleotide molecule, ribozyme, antisense molecule, co-suppression molecule, structural gene, wherein said nucleic acid molecule is introduced into the genome of a cell as an addition to the complement of genetic material present in said cell in the absence of said nucleic acid molecule. In the present context, a transgene is generally integrated into one or more chromosome(s) of the cell, until it is excised therefrom according to the performance of the present invention.

The term “oligonucleotide” refers to any polymer comprising the nucleotides adenine, cytidine, guanine, thymidine, or inosine, or functional analogues or derivatives thereof, capable of being incorporated into a polynucleotide molecule.

The term “synthetic oligonucleotide” refers to any oligonucleotide as hereinbefore defined which is produced by synthetic means, whether or not it is provided directly from said synthetic means.

Those skilled in the art will be aware that the term “ribozyme” refers to a synthetic RNA molecule which comprises a hybridising region complementary to two regions, each of at least 5 contiguous nucleotide bases in the target sense mRNA. In addition, ribozymes possess highly specific endoribonuclease activity, which autocatalytically cleaves the target sense mRNA. A complete description of the function of ribozymes is presented by Haseloff and Gerlach (1988) and contained in International Patent Application No. WO89/05852. The present invention extends to ribozymes which target any sense mRNA, thereby hybridising to said sense mRNA and cleaving it, such that it is no longer capable of being translated to synthesise a functional polypeptide product, subject tot he proviso that said ribozyme is contained within a genetic construct according to any embodiment described herein.

An “antisense molecule” is an RNA molecule which is transcribed from the complementary strand of a nuclear gene to that which is normally transcribed to produce a “sense” mRNA molecule capable of being translated into a polypeptide or peptide sequence. The antisense molecule is therefore complementary to the sense mRNA, or a part thereof. Although not limiting the mode of action of the antisense molecules of the present invention to any specific mechanism, the antisense RNA molecule possesses the capacity to form a double-stranded mRNA by base pairing with the sense mRNA, which may prevent translation of the sense mRNA and subsequent synthesis of a polypeptide gene product.

“Co-suppression” as used herein refers to the reduction in expression of an endogenous gene in a cell that occurs when one or more copies of said gene, or one or more copies of a substantially similar gene are introduced into the cell, regardless of whether or not said endogenous gene is integrated into the chromosome(s) of the cell or maintained as an episome or plasmid therein.

The term “co-suppression molecule” shall be taken to refer to any isolated nucleic acid molecule which is used to achieve co-suppression of an endogenous gene in a cell as hereinbefore defined.

The term “transgenic organism” shall be taken to refer to any organism that has a transgene as hereinbefore defined introduced into its genome.

The term “selectable marker gene” shall be taken to refer to any gene as hereinbefore defined, the expression of which in a cell may be utilised to detect and/or select for the presence of a transgene to which said selectable marker gene is linked or which said selectable marker gene has been co-transformed.

The term “reporter gene” shall be taken to refer to any gene which, when expressed, produces a polypeptide or enzyme capable of being assayed, for example the bacterial chloramphenicol acetyltransferase gene, β-glucuronidase gene and firefly luciferase gene, amongst others. Those skilled in the art will be aware that the coding region of a reporter gene may be placed in operable connection with a promoter sequence such that expression of said reporter gene may be monitored to determine the pattern of expression regulated by said promoter sequence.

As used herein, the terms “hormone gene”, “hormone-biosynthesis gene”, “hormone-encoding gene”, “genetic sequence which encodes a polypeptide capable of regulating hormone levels” or similar term, shall be taken to refer to any gene as hereinbefore defined, in particular a structural gene, which encodes a polypeptide hormone molecule, or alternatively, a gene or structural gene which, when expressed, produces a polypeptide which comprises an enzymatic activity which synthesizes a hormone molecule or a precursor molecule thereof.

As used herein, the term “hormone” encompasses any chemical substance secreted by an endocrine gland of an animal or any plant growth regulatory substance such as, but not limited to, auxins, cytokinins, ethylene, gibberellins, abscisic acid, steroids, prostaglandins, oestrogens, testosterone and progesterones, amongst others.

The term “expression cassette” as used herein refers to a nucleic acid molecule comprising one or more genetic sequences or genes suitable for expression in a cell such as a eukaryotic or prokaryotic cell. In its present context, an expression cassette is particularly preferred to be suitable for expression in a eukaryotic cell such as a plant, animal or yeast cell. In a most particularly preferred embodiment, an expression cassette is suitable for expression in a plant cell.

As used herein, the term “recombinase genetic unit” shall be taken to refer to any genetic sequence which comprises a recombinase gene in a format suitable for constitutive or inducible expression in a cell.

Hereinafter the term “recombinase gene” shall be taken to refer to a gene as hereinbefore defined which comprises a sequence of nucleotides which encodes or is complementary to a sequence of nucleotides which encodes a site-specific recombinase enzyme or polypeptide having site-specific recombinase activity.

A “site-specific recombinase” is understood by those skilled in the relevant art to mean an enzyme or polypeptide molecule which is capable of binding to a specific nucleotide sequence, in a nucleic acid molecule preferably a DNA sequence, hereinafter referred to as a “recombination locus” and induce a crossover event in the nucleic acid molecule in the vicinity of said recombination locus. Preferably, a site-specific recombinase will induce excision of intervening DNA located between two such recombination loci.

The terms “recombination locus” and “recombination loci” shall be taken to refer to any sequence of nucleotides which is recognized and/or bound by a site-specific recombinase as hereinbefore defined.

As used herein the term “transgene unit” shall be taken to refer to any genetic sequence which comprises a transgene as hereinbefore defined, in particular a structural gene selected from the list comprising reporter gene, selectable marker gene, hormone biosynthesis gene or hormone-encoding gene or a genetic sequence which encodes a polypeptide capable of regulating hormone levels, or a ribozyme, antisense molecule, co-suppression molecule or other nucleic acid molecule.

According to this embodiment of the present invention, it is preferred that the recombinase genetic unit comprise a genetic sequence which encodes a site-specific recombinase placed upstream or 5′ of a terminator sequence and operably under the control of a first promoter sequence.

The term “terminator” refers to a DNA sequence at the end of a transcriptional unit which signals termination of transcription. Terminators are 3′-non-translated DNA sequences containing a polyadenylation signal, which facilitates the addition of polyadenylate sequences to the 3′-end of a primary transcript. Terminators active in plant cells are known and described in the literature. They may be isolated from bacteria, fungi, viruses, animals and/or plants. Examples of terminators particularly suitable for use in the genetic constructs of the present invention include the nopaline synthase (NOS) gene terminator of Agrobacterium tumefaciens, the terminator of the Cauliflower mosaic virus (CaMV) 35S gene, the zein gene terminator from Zea mays, the ribulose-1, 5-biphosphate carboxylase small subunit gene (rbcS 1a) terminator, and the isopentenyladenine transferase (ipt) terminator, amongst others.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e. upstream activating sequences, enhancers and silencers) which alter gene expression in response to developmental and/or external stimuli, or in a tissue-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene.

In the present context, the term “promoter” is also used to describe a synthetic or fusion molecule, or derivative which confers, activates or enhances expression of a structural gene or recombinase gene in a cell, in particular a plant cell. Preferred promoters may contain additional copies of one or more specific regulatory elements, to further enhance expression of the gene and/or to alter the spatial expression and/or temporal expression. For example, regulatory elements which confer copper inducibility may be placed adjacent to a heterologous promoter sequence driving expression of a structural gene or recombinase gene, thereby conferring copper inducibility on the expression of said gene.

Placing a gene operably under the control of a promoter sequence means positioning the said gene such that its expression is controlled by the promoter sequence. Promoters are generally positioned 5′ (upstream) to the genes that they control. In the construction of heterologous promoter/structural gene combinations it is generally preferred to position the promoter at a distance from the gene transcription start site that is approximately the same as the distance between that promoter and the gene it controls in its natural setting, i.e., the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

Examples of promoters suitable for use in genetic constructs of the present invention include viral, fungal, animal and plant derived promoters. In a particularly preferred embodiment, the promoter is capable of conferring expression in a eukaryotic cell, especially a plant cell. The promoter may regulate the expression of a gene constitutively, or differentially with respect to the tissue in which expression occurs or, with respect to the developmental stage at which expression occurs, or in response to external stimuli such as physiological stresses, or plant pathogens, or metal ions, amongst others. Examples of preferred promoters according to the present invention include, but are not limited to the CaMV 35S promoter, NOS promoter, octopine synthase (OCS) promoter, Sc1 promoter or Sc4 promoter from subterranean clover stunt virus, seed-specific promoter such as the vicillin promoter or a derivative thereof, floral-specific promoter such as apetala-3, anther-specific promoter, tapetum-specific promoter, root-specific promoter, leaf-specific promoter such as the Arabidopsis thaliana rbcS 1a promoter or other rbcS promoter sequence, stem-specific promoter, light-inducible promoter such as the, Arabidopsis thaliana rbcS 1a promoter or other rbcS promoter sequence, metal-inducible promoter such as the copper-inducible promoter, heat-shock promoter or other environmentally-inducible promoter such as those induced by anaerobiosis or hypoxia or wound-inducible promoter, amongst others. Those skilled in the art will recognise that the choice of promoter will depend upon the nature of the cell being transformed and when expression of the recombinase, structural gene or other gene contained in the genetic construct of the invention is required.

Those skilled in the art will be aware that, in order for a site-specific recombinase polypeptide or enzyme to function in a eukaryotic cell it must be brought into contact with the substrate molecule upon which it acts (i.e. a nucleic acid molecule such as DNA). Furthermore, it is often desirable to ensure that said recombinase is localised in the nucleus of a eukaryotic cell, for example where the recombinase is required to be expressed in stably-transformed cells where the target DNA upon which the recombinase acts has been incorporated or integrated into the genome of the cell.

Accordingly, the recombinase genetic unit of the genetic construct described herein may be further modified in a particularly preferred embodiment to include a genetic sequence which encodes a nuclear localisation signal placed in-frame with the coding region of the recombinase gene. More preferably, the genetic sequence encoding a nuclear localisation signal is placed in-frame at the 5′-terminus or the 3′-terminus, but most preferably at the 5′-terminus, of the coding region of the recombinase gene.

By “in-frame” means that the genetic sequence which encodes the nuclear localisation signal is in the same open reading frame as the genetic sequence which encodes the recombinase with no intervening stop codons, such that when the transcript of the recombinase genetic unit is translated, a single fusion polypeptide is produced which comprises a sequence of amino acids corresponding to the summation of the individual amino acid sequences of the nuclear localisation signal and the recombinase polypeptides.

In the context of the present invention, the essential feature of the recombinase gene is the structural gene region or a derivative thereof which at least encodes a functional site-specific recombinase enzyme. Accordingly, the structural region of a recombinase gene may be any nucleic acid molecule which is capable of encoding a polypeptide having recombinase activity, optionally further comprising one or more intron sequences, 5′-untranslated sequence or 3′-untranslated sequence.

Preferred recombinase genes according to the present invention include the cre gene (Abremski et al, 1983) and flp gene (Golic et al, 1989; O'Gorman et al, 1991). In a particularly preferred embodiment of the present invention, the recombinase gene is the cre gene or a homologue, analogue or derivative thereof which is capable of encoding a functional site-specific recombinase.

The relative orientation of two recombination loci in a nucleic acid molecule or genetic construct may influence whether the intervening genetic sequences are deleted or excised or, alternatively, inverted when a site-specific recombinase acts thereupon. In a particularly preferred embodiment of the present invention, the recombination loci are oriented in a configuration relative to each other such as to promote the deletion or excision of intervening genetic sequences by the action of a site-specific recombinase upon, or in the vicinity of said recombination loci.

Preferred recombination loci according to the present invention are lox and frt, to be used in combination with cre and flp recombinase genes, respectively. Other recombinase/recombination loci systems are not excluded. In a most particularly preferred embodiment, however, the recombination loci are lox sites, such as lox P, lox B, Lox L or lox R or functionally-equivalent homologues, analogues or derivatives thereof.

Lox sites may be isolated from bacteriophage or bacteria by methods known in the art (Hoess et al, 1982). It will also be known to those skilled in the relevant art that lox sites may be produced by synthetic means, optionally comprising one or more nucleotide substitutions, deletions or additions thereto.

Also according to this embodiment of the present invention, the transgene unit preferably comprises a structural gene which encodes a polypeptide, for example the coding region of a gene, placed upstream or 5′ of a terminator sequence and operably under the control of a second promoter sequence.

The terminator and promoter sequences may be any terminator or promoter referred to supra or exemplified herein, amongst others.

The structural gene of the genetic construct of the invention may be any structural gene. Preferably, the structural gene is a selectable marker gene, reporter gene, hormone-biosynthesis gene, hormone-encoding gene or a genetic sequence which encodes a polypeptide capable of regulating hormone levels.

Preferred reporter genes are those genes for which their expression is capable of being assayed, for example the bacterial chloramphenicol acetyl transferase (CAT) gene, bacterial β-glucuronidase (uidA, GUS or gusA) gene, firefly luciferase (luc) gene, green fluorescent protein (gfp) gene or other gene which is at least useful as an indicator of expression.

Preferred selectable marker genes include genes which when expressed are capable of conferring resistance on a cell to a compound which would, absent expression of said selectable marker gene, prevent or slow cell proliferation or result in cell death. Preferred selectable marker genes contemplated herein include, but are not limited to antibiotic-resistance genes such as those conferring resistance to ampicillin, Claforan, gentamycin, G-418, hygromycin, kanamycin, neomycin, spectinomycin, tetracycline or a derivative or related compound thereof or alternatively, herbicide-resistance genes such as those conferring resistance to the compounds atrazine, Basta, bialaphos, bromoxinol, Buctril, 2,4-D, glyphosate, phosphinothricin, suphonylurea or a derivative or related compound thereof, amongst others. The compound names “Basta”, “Buctril”, “claforan” and “G-418” are trademarks.

In a particularly preferred embodiment, the selectable marker gene is the neomycin phosphotransferase gene npt II, which when expressed confers resistance on a cell to neomycin and kanamycin and related compounds thereof. More preferably, the nptII selectable marker gene is placed operably under the control of a promoter suitable for expression in a plant cell.

Preferred hormone-biosynthesis genes, hormone-encoding genes or genetic sequences which encodes one or more polypeptides capable of regulating hormone levels are those genes which encode a polypeptide or enzyme which is involved in at least one biosynthetic step which leads to the production of a plant growth regulatory substance, or at least encode a regulatory polypeptide which is capable of altering the levels of a plant growth regulatory substance in a plant cell.

More preferably, the hormone-biosynthesis or hormone-encoding gene or genetic sequence which encodes a polypeptide capable of regulating hormone levels of the invention, encodes a polypeptide or enzyme which catalyses at least one biosynthetic step leading to the production of a plant growth regulatory substance selected from the list comprising auxins, gibberellins, cytokinins, abscisic acid and ethylene, amongst others, or alternatively, encodes a polypeptide which is capable of altering the levels of one or more of said plant growth regulatory substances in a plant cell.

In a particularly preferred embodiment of the invention, the hormone-biosynthesis or hormone-encoding gene or genetic sequence which encodes a polypeptide capable of regulating hormone levels is a cytokinin gene, more particularly the isopentenyladenine transferase or ipt gene. Genetic constructs comprising the ipt gene are described herein as “Example 9”.

For the present purpose, homologues of a genetic sequence, in particular a structural gene, recombinase gene or recombination locus, shall be taken to refer to an isolated nucleic acid molecule which is substantially the same as, or is functionally identical to, a nucleic acid molecule of the present invention or its complementary nucleotide sequence, notwithstanding the occurrence within said sequence, of one or more nucleotide substitutions, insertions, deletions, or rearrangements.

“Analogues” of a genetic sequence, in particular a structural gene, recombinase gene or recombination locus shall be taken to refer to an isolated nucleic acid molecule which is substantially the same as, or is functionally identical to, a nucleic acid molecule of the present invention or its complementary nucleotide sequence, notwithstanding the occurrence of any non-nucleotide constituents not normally present in said isolated nucleic acid molecule, for example carbohydrates, radiochemicals including radionucleotides, reporter molecules such as, but not limited to DIG, alkaline phosphate or horseradish peroxidase, amongst others.

“Derivatives” of a nucleotide sequence, in particular a structural gene, recombinase gene or recombination locus shall be taken to refer to any isolated nucleic acid molecule which contains significant sequence similarity to said sequence or a part thereof. Generally, the nucleotide sequence of the present invention may be subjected to mutagenesis to produce single or multiple nucleotide substitutions, deletions and/or insertions. Nucleotide insertional derivatives of the nucleotide sequence, of the present invention include 5′ and 3′ terminal fusions as well as intra-sequence insertions of single or multiple nucleotides or nucleotide analogues. Insertional nucleotide sequence variants are those in which one or more nucleotides or nucleotide analogues are introduced into a predetermined site in the nucleotide sequence of said sequence, although random insertion is also possible with suitable screening of the resulting product being performed. Deletional variants are characterised by the removal of one or more nucleotides from the nucleotide sequence. Substitutional nucleotide variants are those in which at least one nucleotide in the sequence has been removed and a different nucleotide or nucleotide analogue inserted in its place.

In an alternative preferred embodiment of the present invention, there is provided a genetic construct comprising a first expression cassette which contains a recombinase genetic unit linked to a transgene unit as hereinbefore defined, wherein said expression cassette is flanked by two recombinant loci upstream and downstream thereof and wherein said recombinase genetic unit further comprises the coding region of a cre gene or a homologue, analogue or derivative thereof and said recombination loci are further defined as loxP sites or a homologue, analogue or derivative thereof.

In a further alternative preferred embodiment, the present invention provides a genetic construct comprising a first expression cassette which contains a recombinase genetic unit linked to a transgene unit as hereinbefore defined, wherein said first expression cassette is flanked by two recombinant loci upstream and downstream thereof and wherein said recombinase genetic unit further comprises a genetic sequence which encodes a nuclear localisation signal placed in-frame with the coding region of a cre gene or a homologue, analogue or derivative thereof and said recombination loci are further defined as loxP sites or a homologue, analogue or derivative thereof.

Preferably, the nuclear localisation signal is the SV40 T-antigen type nuclear localisation signal described by Kalderon et al (1984).

Those skilled in the art will be aware of how to produce the genetic construct of the invention and of the requirements for obtaining the expression thereof, when so desired, in a specific cell or cell-type under the conditions desired. In particular, it will be known to those skilled in the art that the genetic manipulations required to perform the present invention may require the propagation of the genetic construct described herein or a derivative thereof in a prokaryotic cell such as an E. coli cell.

To prevent premature excision events, the recombinase gene of the invention should preferably not be expressed to produce a functional recombinase enzyme during these propagation steps and in any case, until so desired. For example, the recombinase gene may be selected or modified such that it is not expressed in a prokaryote cell, for example by modifying codons within the gene to a codon usage not recognised by the prokarote cell.

Means for preventing the expression of a recombinase gene in a prokaryotic cell whilst allowing its expression in a eukaryotic cell include, but are not limited to the use of a specific promoter which is not recognised by prokaryotic DNA-dependant RNA polymerases, the use of a highly-regulated inducible promoter such as a copper-inducible promoter under non-inducing conditions, the insertion of an intron sequence into the coding region of the recombinase gene, or the insertion of spurious stop codons into a structural gene such that the protein is not translated in a prokaryotic cell but may be translated in a eukaryotic tRNA suppressor mutant cell or organism which is capable of inserting an amino acid at positions where said spurious stop codons occur. Such means for preventing expression of genetic sequences in prokaryotic cells are well-known to those skilled in the art. The present invention extends to the use of all means for preventing expression of the recombinase gene in a prokaryotic cell.

Furthermore, expression of the recombinase gene or the production of a functional recombinase enzyme should preferably occur only when so desired in a eukaryotic cell, tissue, organ or organism. For example, wherein the genetic construct of the invention comprises a structural gene which is a selectable marker gene, expression of the recombinase gene will not normally be required until selection of transformed cells or tissue carrying the genetic construct of the invention has taken place. In many such instances where a cell has been transformed with a genetic construct of the present invention and subsequently selected, expression of the recombinase gene will only be required when regeneration of tissues, organs or the whole organism from the transformed cell has commenced or been completed.

In a further example, wherein the transgene of the transgene unit is a hormone-biosynthesis or hormone-encoding gene or a genetic sequence which encodes a polypeptide capable of regulating hormone levels, expression of said trangene preferably promotes a developmental transition in the transformed cell, for example a transition which leads to differentiation or de-differentiation of cells. In plant cells wherein the structural gene encodes a polypeptide which catalyses the biosynthesis of a plant growth regulatory molecule comprising a cytokinin such as isopentenyladenine, expression of said structural gene preferably leads to the initiation of adventitious shoot formation. Alternatively, wherein the structural gene encodes a polypeptide which catalyses the biosynthesis of a plant growth regulatory molecule comprising an auxin such as IAA, expression of said structural gene preferably leads to the initiation of adventitious root formation. In these cases, it is important that expression of the recombinase be delayed, or at least minimised, until the developmental transition has in fact occurred and expression of the transgene is no longer required, expression of the recombinase may be induced, thereby leading to excision of the transgene.

In a further example, wherein the genetic construct of the invention comprises a structural gene which is a reporter gene, expression of the recombinase gene will not normally be required until the detection of cells which express the reporter gene has taken place.

Those skilled in the art will readily be able to determine the appropriate time when expression of the recombinase gene in a transformed cell, tissue, organ or organism is desirable.

Means for preventing the expression of the recombinase gene in a eukaryotic cell, tissue, organ or organism until so desired includes the use of a tissue-specific promoter which is only capable of conferring significant expression on the recombinase gene in regenerated or regenerating tissues, organs or organisms but not in isolated cells or cell masses or undifferentiated cells or cell masses.

Examples of suitable promoters for use in transgenic plant tissues, organs or organisms for limiting the expression of the recombinase gene thereto include a seed-specific promoter such as the vicillin promoter or a derivative thereof, floral-specific promoter such as apetala-3, anther-specific promoter, tapetum-specific promoter, root-specific promoter, leaf-specific promoter such as the Arabidopsis thaliana rbcS 1a promoter or other rbcS promoter sequence or stem-specific promoter, meristem-specific promoter, amongst other promoter sequences.

Additional means for preventing the expression of the recombinase gene in a eukaryotic cell include the use of an inducible promoter sequence to drive expression thereof, such that no significant recombinase activity is detectable until induction of recombinase gene expression has taken place.

Examples of inducible promoter sequences suitable for use in plants which may be used to control recombinase gene expression include, but are not limited to a light-inducible promoter such as the Arabidopsis thaliana rbcS 1a promoter or other rbcS promoter sequence, metal-inducible promoter such as the copper-inducible promoter, heat-shock promoter or other environmentally-inducible promoter such as those induced by anaerobiosis or hypoxia or wound-inducible promoter, amongst others.

The present invention extends to the use of all means for preventing expression of the recombinase gene until so desired in a eukaryotic cell, such as a plant, animal or yeast cell.

Accordingly, in a particularly preferred embodiment of the present invention, the recombinase gene is modified such that significant expression thereof is limited to a plant or animal tissue, organ or organism, but does not occur in prokaryotic cells such as the bacteria E. coli or Agrobacterium tumefaciens or in isolated cells or cell masses or undifferentiated cells or cell masses derived from eukaryotes.

More particularly, said recombinase gene is modified by the insertion of an intron sequence therein, which is not removed from the primary transcript produced in bacterial cells, thereby resulting in the production of an inactive recombinase enzyme in such cells. In contrast, eukaryotic cells do possess the means for correctly processing primary transcripts which contain an intron sequence and, as a consequence, the intron inserted into a recombinase gene according to this embodiment will be removed from the primary transcript thereof, resulting in the expression of an active recombinase enzyme in eukaryotic cells capable of transcribing said recombinase gene.

Even more particularly, said recombinase gene. modified as described herein, is placed under the control of the Arabidopsis thaliana rbcS 1a promoter or the Sc4 promoter.

The genetic construct of the present invention is particularly suitable for the transformation of a eukaryotic cell to introduce novel genetic traits thereto, in addition to the provision of resistance characteristics described herein to herbicides, antibiotics or other toxic compounds. Such additional novel traits may be introduced in a separate genetic construct or, alternatively on the same DNA molecule as the genetic constructs already described herein. Those skilled in the art will recognise the significant advantages, in particular in terms of reduced genetic manipulations and tissue culture requirements and increased cost-effectiveness, of including genetic sequences which encode such additional traits and the first expression cassette described herein, in a single genetic construct.

Accordingly, an alternative embodiment of the present invention provides a genetic construct comprising:

(i) a first expression cassette which contains a recombinase genetic unit linked to a transgene unit as hereinbefore defined;

(ii) two recombinant loci flanking said first expression cassette; and

(iii) a second expression cassette comprising a transgene for introduction into a eukaryotic cell such as a plant cell or animal cell, wherein said second expression cassette is juxtaposed to one of said recombination loci or separated therefrom by a spacer region of at least 2 nucleotides in length and wherein said second expression cassette is further separated from said first expression cassette.

The distance separating the second expression cassette and the first expression cassette flanked by recombination loci may be varied and, for the present purpose, it is essential only that sufficient distance separate said second expression cassette from said first expression cassette flanked by recombination loci such that, when excision of the expression cassette has taken place, said transgene of the second expression cassette is not also excised.

Preferably, the spacer region is at least 6 nucleotides in length, more preferably at least 10 nucleotides in length and still more preferably at least 50 nucleotides in length.

According to this embodiment, the transgene of the second expression cassette may be any gene as hereinbefore defied, including genes which encode antisense, ribozyme or co-suppression molecules and is not in any way to be limited to a tansgene capable of being translated into a functional enzyme or polypeptide.

In an alternative embodiment, the genetic construct of the present invention is further modified such that the first expression cassette flanked by recombinant loci is inserted into, or embedded within, a second expression cassette which comprises a transgene and terminator laced operably under the control of a promoter sequence, wherein said insertion prevents the expression of the second expression cassette.

The transgene of the second expression cassette may be any transgene as hereinbefore defined. In a particularly preferred embodimend of the invention, the transgene of the second expression cassette is a structural gene, for example a reporter gene, selectable marker gene, hormone-biosynthesis gene or hormone-encoding gene or a genetic sequence which encodes a polypeptide which regulates hormone levels, as hereinbefore defined, or other structural gene sequence.

Preferred reporter genes are selected from the list comprising CAT, GUS, luc or gfp genes, amongst others. Additional transgenes are not excluded. Suitable promoters or terminators are those described previously.

According to this embodiment of the invention, the first expression cassette flanked by recombination loci may be inserted into the second expression cassette at any site which disrupts expression of the transgene of said second expression cassette, such as between the promoter and transgene, or within the transgene sequence.

In a most preferred embodiment, the first expression cassette flanked by recombination loci is inserted between the promoter and the transgene of the second expression cassette.

The present invention extends to all genetic constructs which comprise the specific arrangements of first expression cassette flanked by recombination loci defined herein and additional genes for introduction into a eukaryotic cell and/or expression therein.

In a further embodiment of the present invention, the genetic construct of the present invention is also suitable for integration into the genome of a cell in which it is expressed. Those skilled in the art will be aware that, in order to achieve integration of a genetic sequence or genetic construct into the genome of a host cell, certain additional genetic sequences may be required. For example, the successful integration of DNA into the genome of a plant cell mediated by Agrobacterium tumefaciens requires the presence of one or more left and/or right T-DNA border regions flanking the genetic sequence to be integrated.

Accordingly, the genetic construct of the invention may optionally further comprise additional genetic sequences as required for its integration into the genome of a eukaryotic cell, in particular a plant cell.

Wherein the genetic construct of the invention is intended for use in plants, it is particularly preferred that it be further modified for use in Agrobacterium-mediated transformation of plants by the inclusion of one or more left and/or right T-DNA border sequences. To facilitate Agrobacterium-mediated transformation, the first expression casssette flanked by recombination loci and, where applicable, at least the transgene of the second expression cassette, are usually placed between the left and/or right T-DNA border sequences, if more than one of said sequences is present.

Although intended for the transformation of a eukaryotic organism and/or the expression of genes contained therein, the genetic constructs of the present invention may need to be propagated in a prokaryotic organism such as the bacteria Escherichia coli or Agrobacterium tumefaciens. Accordingly, the genetic constructs described herein may further comprise genetic sequences corresponding to a bacterial origin of replication and/or a selectable marker gene such as an antibiotic-resistance gene, suitable for the maintenance and replication of said genetic construct in a prokaryotic organism. Such sequences are well-known in the art. Usually, an origin of replication or a selectable marker gene suitable for use in bacteria is physically-separated from those genetic sequences contained in the genetic construct which are intended to be expressed or transferred to a eukaryotic cell, or integrated into the genome of a eukaryotic cell.

The present invention extends to all genetic constructs essentially as defined herein, which include further genetic sequences intended for the maintenance and/or replication of said genetic construct in prokaryotes and/or the integration of said genetic construct or a part thereof into the genome of a eukaryotic cell or organism.

The genetic constructs of the present invention are useful in producing genetically-transformed cells and/or for the removal of transgenes from genetically-transformed organisms, in particular eukaryotes such as plants and animals. More particularly, the genetic constructs are used for the transformation of plants with selectable marker genes and/or reporter genes and the subsequent excision in a single-step of said genes.

Accordingly, a further aspect of the present invention provides a method of removing a transgene from a cell transformed with the genetic construct described according to any of the embodiments herein, said method comprising expressing the recombinase genetic unit of said genetic construct for a time and under conditions sufficient for a site-specific recombinase to be expressed and at least excise the first expression cassette of said genetic construct or a fragment thereof sufficient to disrupt expression of the transgene of said first expression cassette.

Preferably, the transgene is a selectable marker gene or a reporter gene or a hormone-biosynthesis gene or hormone-encoding gene or genetic sequence which encodes a polypeptide capable of regulating hormone levels, as hereinbefore defined.

In an alternative embodiment, wherein the transgene of the first expression cassette is to be expressed prior to its excision, this aspect of the invention relates to a method of transiently expressing a transgene in a stably transformed cell, said method comprising:

(i) stably transforming said cell with a genetic construct comprsing a first expression cassette flanked by recombination loci, optionally further comprising a second expression cassette, as described herein;

(ii) expressing the transgene of the first expression cassette in said stably transformed cell; and

(iii) expressing the recombinase genetic unit of said genetic construct for a time and under conditions sufficient for a site-specific recombinase to be expressed and at least excise the first expression cassette of said genetic construct or a fragment thereof sufficient to disrupt expression of the transgene of said first expression cassette.

In a further alternative embodiment, wherein the transgene of the first expression cassette is a structural gene comprising a hormone-biosynthesis gene or hormone-encoding gene or genetic sequence which encodes a polypeptide capable of regulating hormone levels as hereinbefore defied, the expression of which may induce a developmental transition in a cell and/or organogenesis, the genetic construct of the invention may be used to produce a transformed organ. According to this embodiment, the transgene is expressed in a cell transformed with the subject genetic construct, for a time and under conditions sufficient to promote tissue differentiation or organogenesis, or at least the formation of a primordium. Subsequent to this “developmental transition”, and preferably prior to extensive cell division, the recombinase genetic unit of the genetic construct is activated or induced via induction or de-repression of the promoter operably connected to the recombinase gene therein, leading to expression of the site-specific recombinase encoded therefor and subsequent or concomitant recombinase-dependant excision of the transgene. The differentiated cells may be grown or cultured under appropriate conditions to produce a differentiated transformed organ or organism.

Preferred hormone-encoding genes or hormone-biosynthesis genes according to this embodiment include plant growth regulatory substance-encoding genes such as, but not limited to, the ipt gene.

In particular applications of the invention to the production of transformed plants, the genetic construct comprising a plant growth regulatory substance-encoding gene, such as ipt, may be introduced to specific cells of a whole plant, by microinjection or A. tumefaciens-mediated transformation or biolistic methods, wherein expression of the plant growth regulatory substance-encoding gene induces organogenesis in situ, producing a chimeric plant. Alternatively, the genetic construct may also be used to induce organogenesis from undifferentiated cells derived, for example, from a suspension cell culture or callus. Alternatively, the genetic construct according to this embodiment may also be used to induce organogenesis from tissue explant material, for example leaf discs, stem sections, root explants. Those skilled in the art will be aware of the technology requirements for introducing the genetic construct into such plant cells.

As exemplified herein, the inventors have shown that temporary expression of the ipt gene in situ, in plant stem cells, may be used to produce adventitious transgenic shoots on an otherwise untransformed plant.

Similarly, the present invention also contemplates the use of auxin-biosynthesis genes to promote adventitious root formation or gibberellin-biosynthesis genes to promote formation of a floral meristem, amongst others.

This embodiment of the invention is of particular utility to the agriculture and forestry industries, where the regeneration of whole plants from isolated cells may not be efficient or cost-effective and, as a consequence, the production of transformed plants from isolated cells is not a viable or economic proposition. In such cases, the generation of adventitious transformed shoots, roots or other organs may be particularly advantageous, because in vitro regeneration procedures will not be required.

Additionally, the transformed organs may be removed from the parent plant and cultured by micropropagation techniques known to those skilled in the art, to produce a whole transgenic organism.

As in all other embodiments of the invention described herein, the genetic construct may comprise additional genetic sequences which are desired to be permanently maintained in the transgenic organ or transgenic organism, following excision of the hormone-encoding or hormone-biosynthesis gene or genetic sequence which encodes a polypeptide capable of regulating hormone levels. Preferably, these genes are linked to the first expression cassette described herein, but placed outside the recombination loci, or alternatively, flanking said recombination loci such that they are not excised alongside the first expression cassette.

Excision of the first expression cassette contained in the genetic construct of the invention provides a means for the introduction of a second genetic construct comprising the same structural gene or a homologue, analogue or derivative thereof. This is of particular utility where the structural gene encodes a selectable marker gene and it is either undesirable or impractical to produce a transgenic organism which expresses one or more selectable marker genes.

Accordingly, a further aspect of the present invention provides a method for multiply-transforming a cell using a single selectable marker gene, said method comprising the steps of:

(i) transforming said cell with a genetic construct of the invention substantially as previously described, wherein the transgene of the first expression cassette of said genetic construct is a selectable marker gene;

(ii) expressing the recombinase gene contained in said genetic construct in said cell or the progeny of said cell to effect excision of the first expression cassette thereof; and

(iii) transforming the cell obtained in step (ii) with a second genetic construct as hereinbefore described, wherein the transgene of the first expression cassette of said genetic construct is a selectable marker gene which is substantially the same as the selectable marker gene use in step (i) or a homologue, analogue or a derivative thereof.

Optionally, said method comprises the further step of repeating step (ii) above.

Besides marker gene removal and the promotion of organogenesis therein, the inducible excision system described herein has several potential uses.

Firstly, physical methods for plant transformation, including electroporation or CA²+/PEG treatment of protoplasts, biolistic delivery of DNA into plant tissues, or Agrobacterium-mediated plant transformation, often result in multiple tandem insertions, which leads in many cases to transgene instability (Matzke and Matzke, 1995). By placing loxP sites close to the T-DNA boundaries, and linking excision with reconstitution of a useful gene transcriptional unit, the excision system may be used to excise repeated DNA segments after integration into the plant genome. This would reduce any sequence duplication, thereby preventing transgene instability which arises from DNA methylation, co-suppression/antisense mechanisms or recombination.

Secondly, the approach described herein can, with little modification, be adapted to achieve in plants cell-specific ablation. By expressing the inlscre gene from a promoter with tight cellular and temporal patterns of expression, and by coupling excision with reconstitution of a cryptic lethal gene, ablation of particular cells or tissues can be achieved, enabling the study of cell lineages in situ.

Whilst not wishing to be bound by any theory or mode of action, when the genetic construct of the present invention is inserted into the genome of a eukaryotic cell, in particular a plant cell, expression of any transgene therein may occur, either as constitutive or induced expression. Wherein the transgene of the first expression cassette is a structural gene, in particular a selectable marker gene, such expression facilitates the selection of transformed cells. Wherein the transgene of the first expression cassette is a structural gene, in particular a reporter gene, expression thereof facilitates the detection of cells expressing said reporter gene or other structural gene. The subsequent induced expression of the recombinase gene produces an active recombinase enzyme which is capable of recognising the two flanking recombination loci producing a genetic recombination event thereabouts, resulting in excision of the first expression cassette. As a consequence, the first expression cassette is deleted from the genome of the transformed cell, which no longer expresses the transgene of the first expression cassette, for example a selectable marker gene or reporter gene.

Wherein the first expression cassette is inserted into, or embedded within a second expression cassette comprising a promoter, transgene and terminator to disrupt expression thereof, excision of the first expression cassette restores expression of the second expression cassette, thereby facilitating detection of the excision event.

A further aspect of the present invention provides a cell transformed with a genetic construct of the invention substantially as previously described.

Preferably, the transformed cell is a eukaryotic cell such as a plant, animal or yeast cell. More preferably the cell is a plant cell. In a particularly preferred embodiment, the cell is derived from a plant species which is asexually or clonally propagated. Examples of plants which are particularly suited to the practice of the present invention include, but are not limited to stolon-bearing or tuber-bearing plants such as potatoes, sweet potatoes, jerusalem artichoke, taro or yams, fibre or wood tree crops such as Eucalyptus ssp. or Pinus ssp., aspen, ornamental plants such as gerberas, chrysanthemum, orchids, lilies, roses, fuschias, azaleas carnations, camellias or gardenias, citrus crops such as oranges, lemons, grapefruit, tangerines or limes, fruit tress such as apples or pears, berry fruits such as strawberry, raspberry, loganberry or blackberry, tropical crops such as sugarcane, tobacco, bananas, plantain or pineapples or asparagus, amongst others, in particular plants where the removal of transgenes by sexual recombination means is difficult.

In a particularly preferred embodiment of the present invention, the transformed cell is a tobacco cell.

However, the present invention is also useful for removing unwanted genes from any transformed plant species which is capable of being propagated vegetatively from cuttings, stolons, tubers or by grafting, layering etc., as well as by sexual hybridisation.

Means for introducing recombinant DNA into plant tissue include, but are not limited to, direct DNA uptake into protoplasts (Krens et al, 1982; Paszkowski et al, 1984), PEG-mediated uptake to protoplasts (Armstrong et al, 1990) microparticle bombardment electroporation (Fromm et al., 1985), microinjection of DNA (Crossway et al., 1986), microparticle bombardment of tissue explants or cells (Christou et al, 1988; Sanford, 1988), vacuum-infiltration of plant tissue with nucleic acid, or T-DNA-mediated transfer from Agrobacterium to the plant tissue. Representative T-DNA vector systems are described in the following references: An et al. (1985); Herrera-Estrella et al. (1983a,b); Herrera-Estrella et al. (1985).

For microparticle bombardment of cells, a microparticle is propelled into a plant cell, in particular a plant cell not amenable to Agrobacterium mediated transformation, to produce a transformed cell. Wherein the cell is a plant cell, a whole plant may be regenerated from the transformed plant cell. Alternatively, other non-plant cells derived from multicellular species may be regenerated into whole organisms by means known to those skilled in the art. Any suitable ballistic cell transformation methodology and apparatus can be used in practicing the present invention. Exemplary apparatus and procedures are disclosed by Stomp et al. (U.S. Pat. No. 5,122,466) and Sanford and Wolf (U.S. Pat. No. 4,945,050). When using ballistic transformation procedures, the genetic construct may incorporate a plasmid capable of replicating in the cell to be transformed.

Examples of microparticles suitable for use in such systems include 1 to 5 μm gold spheres. The DNA construct may be deposited on the microparticle by any suitable technique, such as by precipitation.

Plant species may also be transformed with the genetic construct of the present invention by the DNA-mediated transformation of plant cell protoplasts and subsequent regeneration of the plant from the transformed protoplasts in accordance with procedures well known in the art.

Any plant tissue capable of subsequent clonal propagation, whether by organogenesis or embryogenesis, may be transformed with a vector of the present invention. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The term “organogenesis”, as used herein, means a process by which shoots and roots, or other organs, are developed sequentially from meristematic centers.

The term “embryogenesis”, as used herein, means a process by which shoots and roots develop together in a concerted fashion (not sequentially), whether from somatic cells or gametes.

Plants of the present invention may take a variety of forms. The plants may be chimeras of transformed cells and non-tansformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, a first generation (or T1) transformed plants may be selfed to give homozygous second generation (or T2) transformed plants, and the T2 plants further propagated through classical breeding techniques.

Following excision of the first expression cassette of the genetic construct defined herein, a small “footprint” may be left in the genome of the transformed cell.

As used herein, the term “footprint” shall be taken to refer to any derivative of a genetic construct described herein which is produced by excision, deletion or other removal of the first expression cassette from the genome of a cell transformed previously with said genetic construct.

A footprint generally comprises at least a single copy of the recombination loci used. However, a footprint may comprise additional sequences derived from the genetic construct, for example nucleotide sequences derived from the recombinase gene unit, left border sequence, right border sequence, first expression cassette, second expression cassette, origin of replication, or other vector-derived nucleotide sequences. More likely, a footprint will comprise, in addition to the single copy of a recombination locus, nucleotide sequences derived from the recombinase gene unit, transgene unit of the first expression cassette, or other first expression cassette sequences.

Accordingly, a footprint is identifiable according to the nucleotide sequence of the recombination locus of the genetic construct. In particular, the footprint will comprise a sequence of nucleotides corresponding or complementary to a lox site.

A footprint thus comprises a sequence of at least about 30 nucleotides, preferably about 40 nucleotides, more preferably at least about 50 nucleotides and even more preferably at least about 100 nucleotides derived from the sequences outside (i.e. upstream and downstream) the region of the second expression cassette.

Those skilled in the art will readily be capable of determining whether a cell comprises a footprint of a genetic construct of the invention as hereinbefore defined, using known techniques and without undue experimentation.

Accordingly, the present invention extends to a transformed cell or whole organism which comprises a footprint derived from a genetic construct as hereinbefore defined and to the progeny of said transformed cell or whole organism.

The present invention is flirter described with reference to the following non-limiting Figures and Examples.

### IN THE FIGURES

FIG. 1 is a schematic representation of the cre/lox site-specific recombination constructs; (A) Site-specific recombination test sequences in plasmid pBS210, and pBS210a, the predicted product of recombination. In pBS210, the EcoRI-HindIII fragment containing the Sc4 promoter (Sc4), a 35Spromoter-nptII-35S3′ transcriptional unit (nptII) flanked by loxP (lox) sites (arrowhead) in direct-repeat configuration, and a promoterless gusA-nos3′ cassette, is shown. cre/lox site-specific recombination should remove the loxP-bound nptII transcriptional unit, producing pBS210a. Restriction enzyme designations: E, EcoRI; H, HindIII. (B) T-DNA regions of the binary vectors pBS215 and its derivative, pBS229. pBS215 contains the EcoRI-HindIII fragment from pBS210 between the T-DNA left (LB) and right border (RB) sequences. In pBS229, a rbcS 1a promoter-inlscre-rbcS 1a3′ cassette (inlscre) was cloned into the XhoI (X) site of pBS215. Arrows in boxes indicate the direction of transcription.

FIG. 2: is a photographic representation showing histochemical staining for GUS activity. 2 ½-week old regenerating tobacco calli were stained for GUS activity using X-gluc. Blue coloration indicative of GUS activity is seen, usually localised but in some cases throughout the regenerating shoot.

FIG. 3 is a photographic representation of a ³²P-labelled autoradiogram showing neomycin phosphotransferase (NptII) activity assays. Extracts of two leaves from each plant were assayed for NptII activity, and 15 μl of the reaction blotted onto Whatman P-81 paper. The plant from which the extract was derived is shown (numbers) at the top left corner of each pair of spots. Shown are the NptII activity dot blots for five ntBS229 GUS⁺T₀ plants (#4,7,8,17 and 20), and one GUS⁻ plant (#6) (FIG. 3A), and for thirteen ntBS229-4 regenerants (FIG. 3B). Included are the activities corresponding to positive (+) and negative (−) controls.

FIG. 4a is a schematic representation of the genomic copies of the pBS229 T-DNA construct carried by ntBS229 plants before (panel A) and after the predicted cre/lox-mediated site-specific recombination event (panel B). Indicated below each map are the primers (triangles A-E) used for PCR analysis of DNA prepared from these plants. The expected PCR product obtained using each of the primer pairs indicated is represented as a line with the expected size (kb) of the PCR product shown below.

FIG. 4b is a photographic representation showing the results of the PCR analysis for ntBS229 T₀ and ntBS229-4 regenerated plants (lanes 1-6), with the primers used in each case indicated above the numbered lanes. Template DNA was isolated from either a chimeric Gus⁺nptII⁺ T₀ plant, ntBS229-4 (lanes 1,3,5) or from a typical GUS nptII ntBS229-4 regenerant (lanes 2,4,6). Lane S, EcoRI-digested SPP1 DNA and HpaII-digested pUC19 size markers.

FIG. 5 is a schematic representation of the cre/lox site-specific recombination binary vector plasmids pBS266 and pBS267. Each plasmid contains the Sc4 promoter (Sc4), a cre and an Sc1 promoter-nptII-Sc3 terminator (Sc1-NptII) cassette both flanked by loxP (P) sites in direct repeat configuration, and a promoterless gusA-nos3′ cassette. The cre cassette present in pBS266 is pAp1-inlscre-nos3′ (pAp1-inslcre), while in pBS267 it is pVic-inlscre-nos3′ (pVic-inlscre). With both pBS266 and pBS267, cre/lox site-specific recombination should remove the cre and Sc1-nptII cassettes, producing a transcriptionally active Sc4 promoter-driven gusA transcriptional unit, as shown. Arrows in boxes indicate the direction of transcription, while the dotted lines represent the T-DNA left border (Lb) and right border (Rb).

FIG. 6 is a schematic representation of relevant parts of the ipt constructs and related plasmids. In pRDF9574, the HindIII fragment containing an enhanced 35S promoter (e35S), tobacco mosaic virus 5′ untranslated region (TMV5′), NcoI and BamHI restriction sites and nos3′ termination region is shown. To make pRDF10072, the NcoI-BamHI fragment from pRZ4 was inserted between the NcoI and BamHI sites of pRDF9574. To make pRDF10086, the HindIII fragment from pRDF10072 containing the ipt gene was inserted into the HindIII site of the binary vector pIG121-Hm (Hiei et al, 1994), between the T-DNA left (LB) and right border (RB) sequences. Arrows in boxes indicate the direction of transcription. Restriction site designations: H, HindIII; N, NcoI; B, BamHI.

FIG. 7 is a schematic representation of relevant parts of plasmids used to construct pRDF10543. In pBS209, an EcoRI-HindIII fragment is shown containing the Sc4 promoter (Sc4), loxP (lox) sites (large arrowheads) in direct-repeat configuration, XbaI and XhoI restriction sites, and a gusA-nos3′ cassette. Several changes were made to pBS209 as described in Example 2 to make pRDF10501, including introduction of an intron into the gusA coding region (introngusA). This HindIII fragment was inserted into pRDF10346, a binary vector containing nptII (nos-npt-nos3′) and oxy (35S-oxy-nos3′) genes between the T-DNA left (LB) and right border (RB) sequences, to make pRDF10543. Arrows in boxes indicate the direction of transcription. Restriction site designations: H, HindIII; E, EcoRI; Xba, XbaI; Xho, XhoI.

FIG. 8 is a schematic representation of a genetic construct containing an excisable ipt gene. The 35S-ipt-ipt3′ gene is inserted into the XbaI site of pRDF10543, and the product is used for insertion of the ssu-inlscre-ssu3′ fragment from prbcS-inlscre. All other designations are as for FIG. 7. Excision of the 35S-ipt-ipt3′ and SSU-inlscre-ssu3′ transgenes via cre-mediated recombination at lox sites leads to reconstitution of gusA gene expression under the control of the Sc4 promoter in transformed plant cells.

FIG. 9 is a copy of a photographic representation of a ³²P-labelled autoradiogram showing neomycin phosphotransferase (Npt) activity assays. Extracts of leaves from 17 shoots (numbers 1-17) that arose after inoculation of tobacco plants with Agrobacterium AGL1/pRDF10086 or from control, untransformed leaves (C) were assayed for Npt activity according to McDonnell et al, (1987). Shoot Nos. 4, 5, 9, 15, 16, and 17 were clearly positive for Npt activity.

FIG. 10 is a photographic representation of a shoot (arrow) that arose on a tobacco plant after inoculation with Agrobacterium AGL1/pRDF10086. The shoot had a stem that was pale green to white in colour, with thickened leaves and stems, showed obvious loss of apical dominance, and was phenotypically Gus-positive and Npt-positive. The shoot was approximately 10 cm long 9 weeks after inoculation.

FIG. 11 is a photographic representation of a shoot, (arrow) approximately 2 cm long, that arose on a tobacco plant after inoculation with Agrobacterium AGL1/pRDF10086. The shoot was mostly creamy white in colour with distinct zones of normal green colour. The white zones were Gus-positive, the green zones were Gus-negative.

FIG. 12 is a photographic representation of a cluster of shoots (arrow) approximately 2 cm long, that arose on a tobacco plant after inoculation with Agrobacterium AGL1/pRDF10086. The shoots were normal green in colour and phenotypically Gus-negative and Npt-negative.

### EXAMPLE 1

### Enzymes and Chemicals.

Restriction enzymes, DNA polymerase I large fragment (Klenow) and T₄DNA ligase were purchased from New England Biolabs, and AmpliTaq DNA polymerase from Perkin Elmer. Kanamycin sulfate was purchased from Sigma, and 5-bromo4-chloro-3-indolyl-β-D-glucuronic acid (X-gluc) was from Diagnostic Chemicals (Canada). Oligonucleotides were synthesised on an Applied Biosystems, 394 DNA synthesiser.

### EXAMPLE 2

### Plasmid Constructions.

Cloning and related techniques were performed essentially as described by Sambrook et al (1989) with minor variations. Nucleotide sequences of plasmid constructs were verified by DNA sequencing of plasmid DNA using the dideoxy chain-termination method (Sanger et al, 1977).

(i) Construction of pUC119-cre, pUC119-nlscre and pUC119-inlscre.

The cre open reading frame (orf) was amplified by polymerase chain reaction (PCR) from the bacteriophage P1 genome using the 5′ cre and 3′ cre oligonucleotide primers (primers D and E, respectively set forth in Example 4). Using these primer sequences, an NcoI site was introduced at the initiating ATG of the cre orf, resulting in a Ser −> Ala change in the amino acid sequence of the cre polypeptide, at amino acid position 2. The amplified DNA fragment was digested with EcoRI and cloned into the EcoRI site of pUC119 (Vieira and Messing, 1987), creating pUC119-cre, for subsequent modification.

An SV40 T-antigen type nuclear localisation signal (nls), comprising the amino acid sequence Met-Ala-Pro-Lys-Lys-Lys-Arg-Lys-Val-Thr (Kalderon et al, 1984), was introduced upstream of the cre coding region in the plasmid pUC119-cre. A double stranded synthetic DNA fragment encoding nls was produced by primer extension using Klenow enzyme and subsequently cloned into the HindIII and NcoI sites of the plasmid pUC119-cre, creating pUC119-nlscre. When translated, the nlscre orf produces an in-frame fusion polypeptide between nls and, cre polypeptides.

The third intron of the Parasponia andersonii haemoglobin gene (Landsmann et al., 1986) was isolated by PCR and inserted, using the PstI termini introduced by the PCR primers, into plasmid pUC119-nlscre, to disrupt the nlscre orf. First, a PstI site was introduced into the acre orf of pUC119-nlscre without altering the amino acid sequence encoded thereby, using site-directed mutagenesis to substitute T for G at position 264 of the nlscre orf (²⁶²CTGCAG). The haemoglobin intron was then cloned as a PstI fragment into the PstI site of pUC119-nlscre, to produce the plasmid pUC119-inlscre.

(ii) Construction of p35S-cre, p35S-nlscre and p35S-inlscre.

The cre, nlscre and inlscre genes were cloned from their respective pUC119 plasmids into pJ35SN (Landsmann et al, 1989), creating the plasmids p35S-cre, p35S-nlscre and p35S-inlscre, respectively. In these plasmids, expression of cre and its derivatives is under control of the cauliflower mosaic virus 35S (35S) promoter. Furthermore, the nopaline synthase gene polyadenylation signal (nos3′) is located downstream of the cre orf in each plasmid.

(iii) Construction of prbcS-inlscre.

The EcoRI fragment of pUC119-inlscre comprising the inlscre orf was end-filled using Klenow enzyme and placed upstream of a 0.45 kb rbcS 1a polyadenylation signal (rbcS 1a 3′ end) and operably under the control of the 1.7 kb A. thaliana rbcS 1a promoter sequence (Donald and Cashmore, 1990) in pWM5 (Tabe et al, 1995). The resulting construct was designated prbcS-inlscre.

(iv) Construction of pBS210

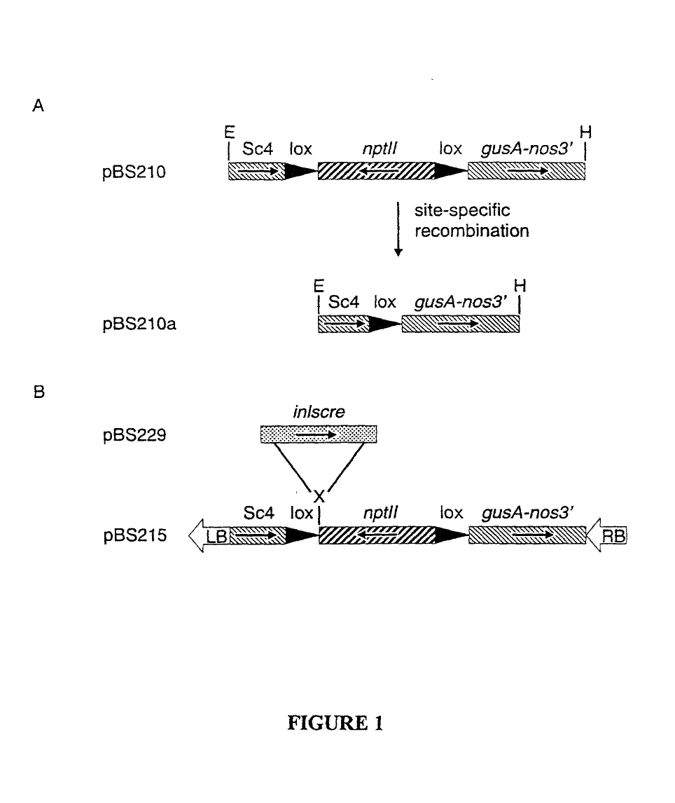

This plasmid, a derivative of the vector pGEM3zf+ (Promega), contained a cryptic gusA reporter gene upstream of the nos3′ polyadenylation signal and placed operably under the control of the Sc4 promoter from the genome of subterranean clover stunt virus (SCSV) (Boevink et al, 1995). A schematic representation of pBS210 is provided in FIG. 1A.

The gusA reporter gene was inactive by the insertion of a DNA fragment containing a loxP-bound neomycin phosphotransferase gene (nptII) expressed from the 35S promoter and 35S polyadenylation signals (35S 3′) (Tabe et al, 1995), between the Sc4 promoter and the gusA coding sequence. Site-specific recombination of pBS210 in which excision of the lox-bound 35S-nptII-35S cassette occurs, produces the plasmid pBS210a (FIG. 1A).

(v) Construction of pBS215 and pBS229.

The Sc4-lox-35S-nptII-35S-lox-gusA-nos cassette was cloned out from the plasmid pBS210 (FIG. 1A) as an EcoRI-HindIII fragment, from upstream of the Sc4 promoter (EcoRI) to downstream of the nos3′ polyadenylation signal (HindIII), end-filled using Klenow enzyme and cloned into the end-filled BamHI and EcoRI sites of the binary vector pTAB5 (Tabe et al, 1995). The new binary vector thus produced was designated pBS215 (FIG. 1B) in which the loxP-bound 35S-nptII-35S cassette provided the only selectable marker.

Plasmid pBS215 contains a unique XhoI site adjacent to the 35S 3′ end of the nptII cassette within the region bounded by loxP. A blunt-ended EcoRI fragment, containing the rbcS 1a promoter placed upstream of the inlscre orf and rbcS 1a 3′ end (i.e rbcS 1a-inlscre-rbcS 1a), was sub-cloned from the plasmid prbcS-inlscre into the end-filled XhoI site of pBS215, creating the plasmid pBS229 (FIG. 1B).

vi) Construction of pRDF10072 and pRDF10086

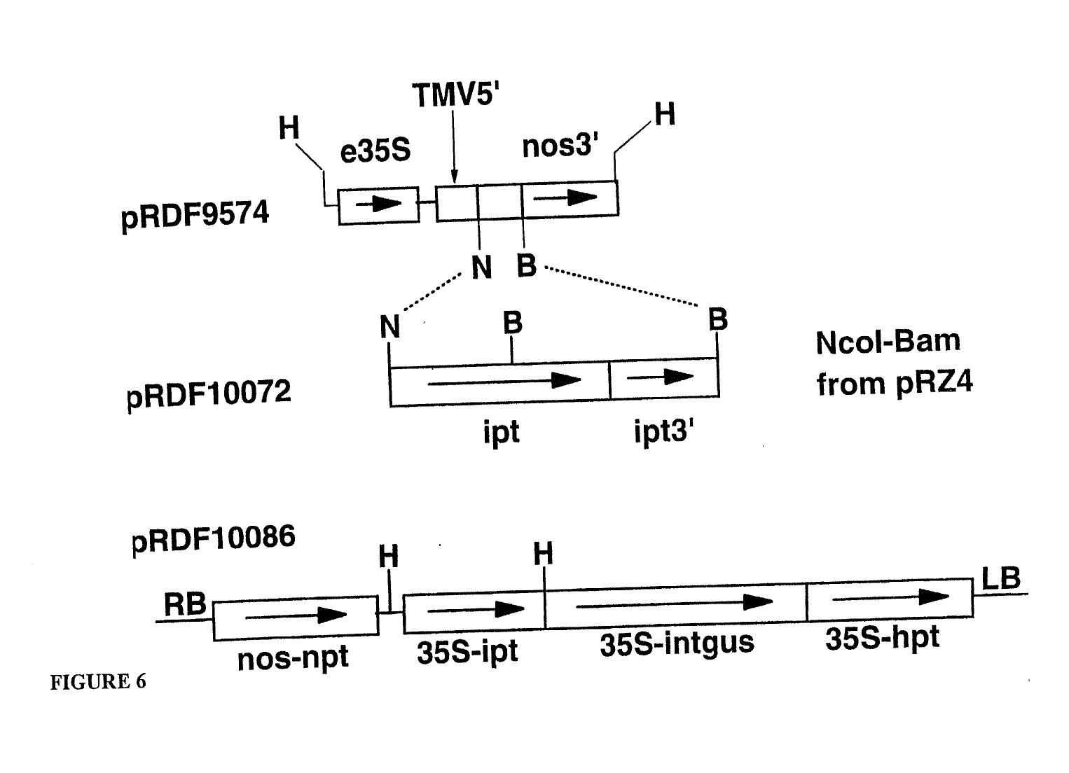

The ipt-ipt3′ cassette was cloned out from plasmid pRZ4, a derivative of pRZ3 (Ma et al, 1997) containing an NcoI site at the translation initiator ATG of ipt, as an NcoI-BamHI fragment (partial digestion with BamHI) and inserted between the NcoI and BamHI sites of pRDF9574 (de Feyter et al, 1997) to create pRDF10072 (FIG. 6). pRDF9574 contains plant gene expression signals including an enhanced 35S promoter (Kay et al, 1987), the tobacco mosaic virus (TMV) 5′ untranslated region corresponding to nucleotides 1-67 of TMV (Goelet et al, 1982) and the 3′ terminator region of a nopaline synthase gene (nos). The HindIII fragment containing the ipt gene of pRDF10072 was inserted into the HindIII site of the binary vector pIG121-Hm (Hiei et al, 1994) to create pRDF10086 (FIG. 6)

(vii) Construction of pRDF10302, pRDF10453 and pRDF10501

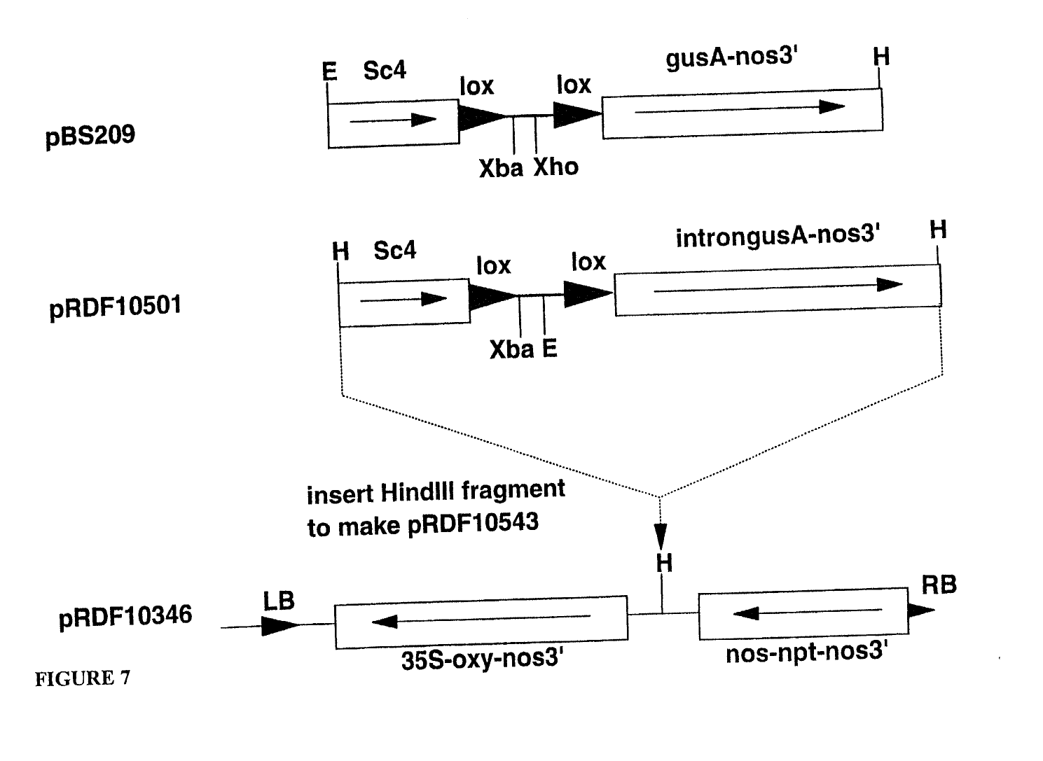

pBS209 is identical to pBS210 (FIG. 1) except that it lacks the nptII gene. pBS209 (FIG. 7) contains an Sc4 promoter and a gusA coding region flanking a pair of lox recombination sites. pBS209 also has unique XhoI and XbaI sites between the lox sites. The EcoRI site of pBS209 was converted to a HindIII site using an EcoHind adaptor (5′ AATTAAGCTT 3′), creating pRDF10302. The Sc4-lox-gusA-nos3′ cassette contained on pRDF10302 conferred Gus activity to Agrobacterium when introduced into the bacterium on a binary plasmid, so an intron was inserted into the gus coding region to prevent Gus expression in bacteria. This was achieved by replacing a ClaI-SnaBI fragment, containing a 5′ portion of the gus coding region, from pRDF10302 with an XbaI-SnaBI fragment from pIG121-Hm (Hiei et al, 1994) containing the corresponding 5′ portion of the gus gene with an intron inserted. The digested ClaI and XbaI ends were endfilled using Klenow enzyme prior to ligation. The resultant plasmid was designated pRDF10453. The S4-lox-introngusA-nos3′ cassette of pRDF10453 directed expression of Gus activity in tobacco cells in transient assays, but did not confer Gus activity to Agrobacterium cells, indicating that insertion of the intron achieved its purpose. An EcoRI site was introduced into pRDF10453 at the position of the XhoI site using an XhoEco adaptor (5′ TCGAGAATTC 3′), creating pRDF 10501 (FIG. 7).

(viii) Construction of pRDF10278 and pRDF10543

A polylinker containing KpnI, SacI and EcoRI sites was deleted from pRPA-BL429, a plasmid containing a 35S-oxy-nos3′ gene provided by Rhone-Poulenc, by digestion with KpnI (partial) and EcoRI followed by blunting with T4 DNA polymerase and recircularisation with T4 DNA ligase, creating pRDF10278. A 2.2 kb HindIII-KpnI fragment of pRDF10278 containing the 35S promoter and oxy coding region, after blunting the digested KpnI end with T4 DNA polymerase, was inserted between the HindIII and BamHI (endfilled) sites of pIG121-Hm, creating pRDF10346 (FIG. 7). The binary vector pRDF10346 contains a nptII gene and an oxy gene (Stalker et al, 1988), driven by nos and 35S promoters, respectively. The HindIII fragment containing the Sc4-lox-introngus-nos cassette from pRDF10501 was inserted into the HindIII site of pRDF10346, creating pRDF10543 (FIG. 7). This plasmid confers Gus expression and resistance to bromoxynil on plant cells.

(ix) Construction of a genetic construct containing an excisable ipt gene

The HindIII fragment containing the 35S-ipt-ipt3′ gene from pRDF10072 is inserted into the XbaI site of pRDF10543 (FIG. 7). This is done readily after half filling the restricted sites, treating the HindIII ends with Klenow, dATP and dGTP, and the XbaI ends with Klenow, dCTP and dTTP, before ligation of the fragments. The resultant plasmid contains a unique EcoRI site which is used for insertion of an EcoRI fragment containing the ssu-inlscre-ssu3′ cassette from prbcS-inlscre, creating a genetic construct that contains excisable ipt and inlscre genes. This construct is then introduced into Agrobacterium for subsequent inoculation into plants.

### EXAMPLE 3

### Protoplast Assays, Transgenic Plants and Phenotype Analysis

Protoplasts of Nicotiana plumbaginifolia were prepared, electroporated with DNA and assayed for β-glucuronidase (GUS) activity as described by Graham and Larkin (1995).

For Agrobacterium-mediated transformation of plant material with the plasmid pBS229, pBS229 was transferred into Agrobacterium tumefaciens strain LBA4404 and leaf discs of Nicotiana tabacum cv. Wisconsin 38 were infected with LBA4404/pBS229 as described by Ellis et al (1987), with the following modifications to the plant transformation procedure. Leaf pieces were co-cultivated with A. Tumefaciens cells containing plasmid pBS229, and maintained in the dark for two weeks on MS medium (Murashige and Skoog, 1962) containing 100 μg/ml kanamycin sulfate and 500 μg/ml cefotaxime (Claforan, Hoechst). The leaf pieces were then transferred to the light, and kept on MS media without antibiotic selection.

The GUS phenotype of transformed plant tissue was determined by histochemical staining with X-gluc (Jefferson et al, 1987). NptII assays were performed on transgenic leaf tissue extract according to (McDonnell et al, 1987).

### EXAMPLE 4

### Molecular Analysis of Plant DNA.

Plant DNA was prepared according to deFeyter (1996).

DNA was amplified in PCR reactions using 30 cycles of denaturation, annealing and extension at 94° C. for 15 sec, 55° C. for 30 sec, and 72° C. for 1 min, respectively. Reaction products were resolved by electrophoresis in 1.5% (w/v) agarose gels.

The sequences of the PCR primers used to analyse plant DNA were as follows:

Primer A:
5′-ATAAGAATGCGGCCGCACCCCGTGCCGGGATCAG-3′;
Primer B:
5′-CATCAGAGCAGCCGATTGTCT-3′;
Primer C:
5′-GGTTTCTACAGGACGTAACAT-3′;
Primer D:
5′-GCGGAATTCGTCGACCATGGCCAATTTACTGACCG-3′;
Primer E:
5′-GCGGAATTCAATCATTTACGCGTTAATGG.

### EXAMPLE 5

### Demonstration of cre/lox-mediated Excision in Transient Expression Assays

The strategy described herein is based upon an improvement to the inducible cre/lox-mediated cis-excision of transgenes, in particular selectable marker genes used in plant transformation.

The Examples described herein report the preparation of a DNA construct carrying the cre gene expressed from a regulatable plant promoter, and a selectable marker gene, nptII, which encodes neomycin phosphotransferase. The cre and nptII transcriptional units are located within the segment of DNA flanked by loxP sequences. In attempts to make a cis-acting excision construct by ligation of the cre gene, or its derivative containing a nuclear localisation signal (nlscre), into a plasmid containing two loxP sites in direct repeat configuration, all recovered recombinant plasmids had deletions consistent with cre/lox-mediated excision (data not shown). To prevent premature excision in E. coli, the third intron of the P. andersonii haemoglobin gene was introduced into the cre coding region of the nlscre orf. This modified orf, inlscre, was able to be cloned into loxP-containing plasmids, indicating that the presence of the intron significantly reduced expression of nlscre in bacteria.

The inlscre orf was then assayed in a recombination test system and its activity compared to that of the cre and nlscre genes, to determine whether inlscre potentially expressed wild-type cre recombinase activity in eukaryotic cells. The recombination substrate in this assay, plasmid pBS210, carries a gusA reporter gene construct rendered inactive by the insertion of the 35S-nptII-35S transcriptional unit between the promoter (Sc4) and the gusA gene (FIG. 1A). The 35S-nptII-35S cassette is bound by two loxP sites in pBS210, in direct-repeat configuration. A successful cre/lox-mediated recombination event should excise the DNA fragment between the two loxP sites, removing the nptII cassette and producing the expected recombination test product, pBS210a (FIG. 1A), thereby activating the Sc4 promoter-derived expression of the gusA gene. The Sc4 promoter drives high level GUS expression in tobacco protoplasts and callus, and predominantly vascular expression in tobacco plants (Boevink et al, 1996).

The recombination mechanism shown in FIG. 1A was tested initially in a transient expression assay using transfected tobacco protoplasts. Protoplasts were electroporated in the presence of plasmid pBS210 alone or co-electroporated with pBS210 plus p35S-cre, pBS210 plus p35S-nlscre or pBS210 plus p35S-inlscre. GUS activity was measured after 72 hours. The results obtained (Table 1) indicate that plasmid PBS210 is unable to express GUS in eukaryotic cells, in the absence of cre. The inclusion of a plasmid capable of expressing cre or nlscre in electroporations activated GUS expression of pBS210. Whilst not wishing to be bound by any theory or mode of action, GUS expression was the result of cre/lox-mediated recombination of pBS210, producing the expected excision product pBS210a (FIG. 1A).

Furthermore, the data shown in Table 1 indicate that the inlscre gene encoded as much as 37% of the recombinase activity of the cre or nlscre genes (Table 1), suggesting that splicing of the intron was occurring in transfected protoplasts. The transient expression data validated the cre/lox-mediated recombination mechanism involving pBS210, shown schematically in FIG. 1A.

A modified version of plasmid pBS210 was prepared for subsequent use in the in planta gene excision experiments described below, in Examples 6-8.

TABLE 1
cre/lox-mediated reconstitution of GUS expression from PBS210

| ELECTROPORATED PLASMID | β-Glucuronidase, units/25 μg protein |
|---|---|
| pBS210 | 0 |
| pBS210 + p35s-cre | 133 |
| pBS210 + p35S-nlscre | 141 |
| pBS210 + p35S-inlscre | 51 |

Protoplast extracts were prepared and β-Glucuronidase activity was measured by the MUG method. Activities (relative fluorescence units) represent the average of two experiments.

### EXAMPLE 6

### Inducible cre-lox Mediated in Planta Gene Excision

To demonstrate the principle of in planta inducible cre/lox-mediated gene excision in cis, a construct was prepared which contained a plant regulatable inlscre transcriptional unit adjacent to the nptII marker gene. As both genes are within the region of DNA bound by loxP, premature expression of nlscre in callus culture would lead to excision of the nptII gene before the selection of transgenic tissue was completed. To avoid this, the inlscre gene was expressed from the rbcS 1a promoter which had low activity in callus culture, and high activity in regenerating or regenerated tissues, organs or organisms. Sequences contained within the 1.7 kb rbcS 1a promoter fragment were previously shown to confer light-inducible expression on a heterologous gene in tobacco (Donald and Cashmore, 1990).

Preliminary experiments showed that no GUS activity could be detected when a construct containing the gusA gene driven by the rbcS 1a promoter and polyadenylation signals (rbcS 1a 3′ end) was introduced into tobacco by Agrobacterium-mediated transformation of leaf discs and subsequent regeneration in the dark for up to 3 weeks. In contrast, GUS expression was apparent in a similar experiment conducted in parallel, wherein a 35S promoter-driven gusA-nos3′ construct was introduced into plant cells (data not shown).

Furthermore, as inlscre had the least activity of the three cre genes tested in the protoplast experiments (Table 1), the inventors considered that use of this gene as source of nlscre would provide an even tighter control of nlscre expression in planta.

The T-DNA region of the plasmid construct pBS229 (FIG. 1B), was introduced into tobacco using Agrobacterium-mediated plant transformation procedures as described above. Since the activity of the rbcS 1a promoter is light-inducible (Donald and Cashmore, 1990), inlscre expression was reduced until desired, by regenerating transgenic ntBS229 tissue initially in the dark, in the presence of kanamycin. This procedure avoided premature nptII excision. After two weeks, calli were transferred to media lacking kanamycin, and regeneration continued under normal light conditions.

### EXAMPLE 7

### Regeneration of Plants Free of the nptII Gene

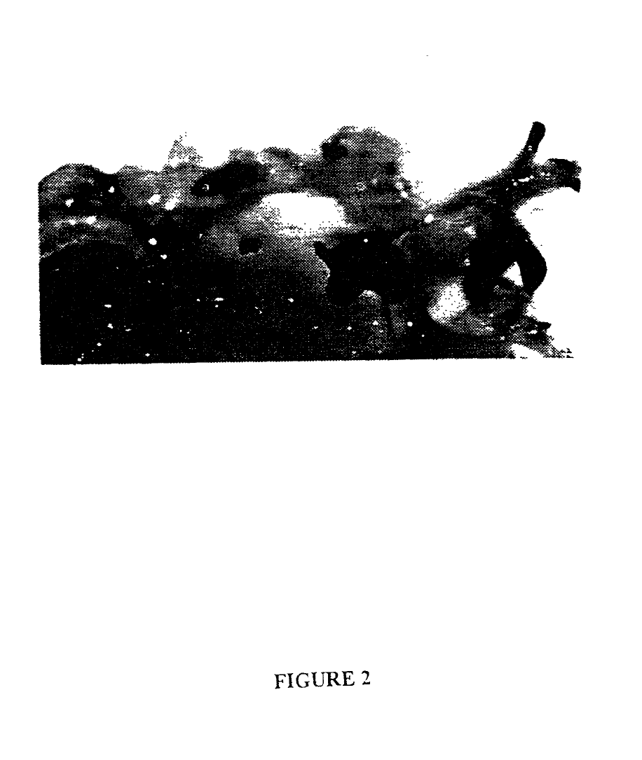

After three days in the light, small pieces of callus with developing shoots were removed and assayed for GUS expression by staining with X-gluc. A proportion of the tested shoots stained blue (FIG. 2), indicating expression of the GUS gene therein. These data suggest that excision of the DNA segment flanked by loxP had occurred in the transformed, regenerating shoots, thereby reconstituting the Sc4-GUS transcriptional unit (FIG. 1A).

One month after continued regeneration in the light without kanamycin selection, leaves were taken from eighteen ntBS229 plants and stained for GUS activity. Five plants showed GUS activity in tissues for which Sc4 promoter-driven GUS expression is normal (not shown).

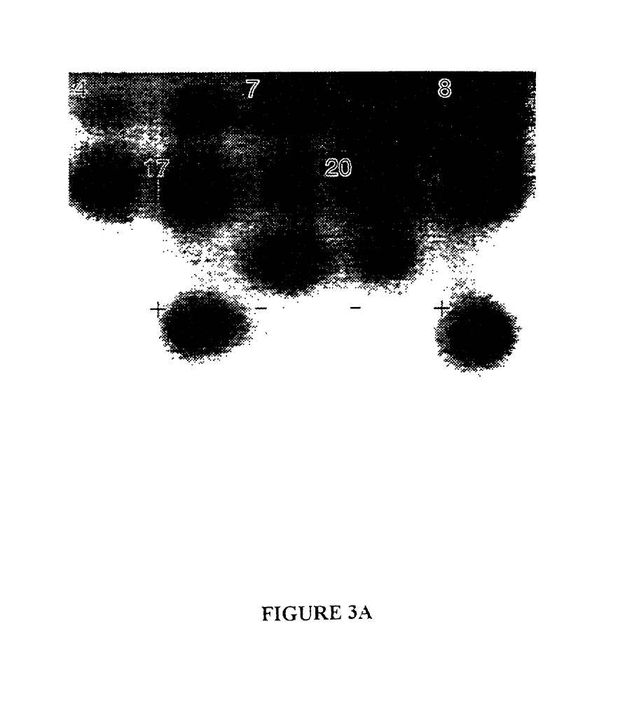

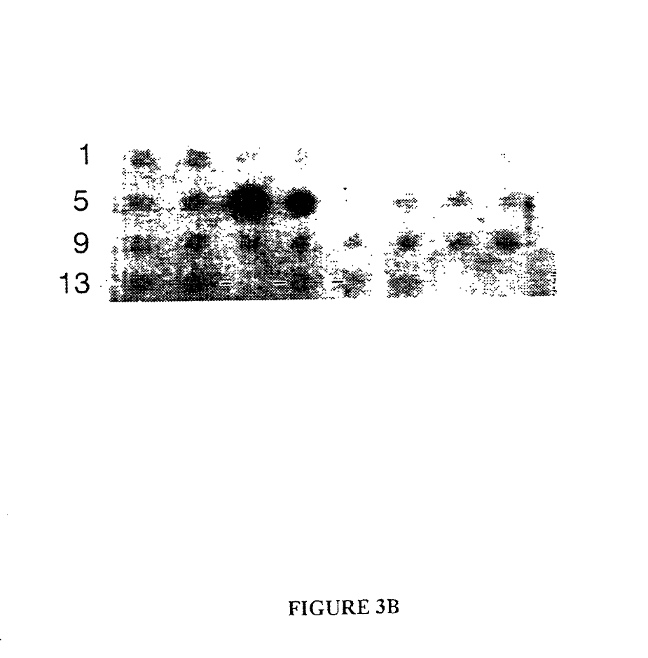

A young leaf and an old leaf were taken from each of the eighteen ntBS229 GUS⁺ plants and from one GUS⁻ plant and assayed for nptII activity. All Gus⁺ and Gus⁻ leaves tested had high nptII activity levels, with the exception of one leaf from plant ntBS229-4 (FIG. 3A).

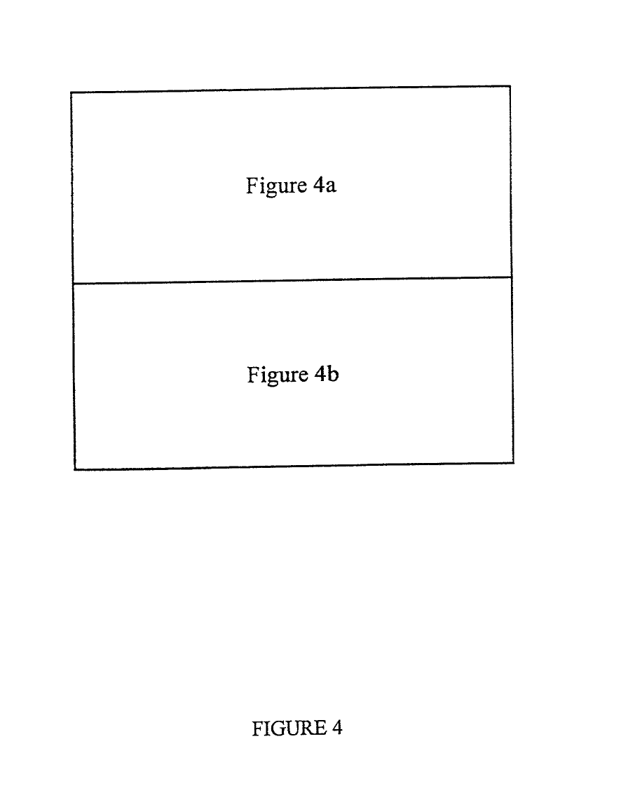

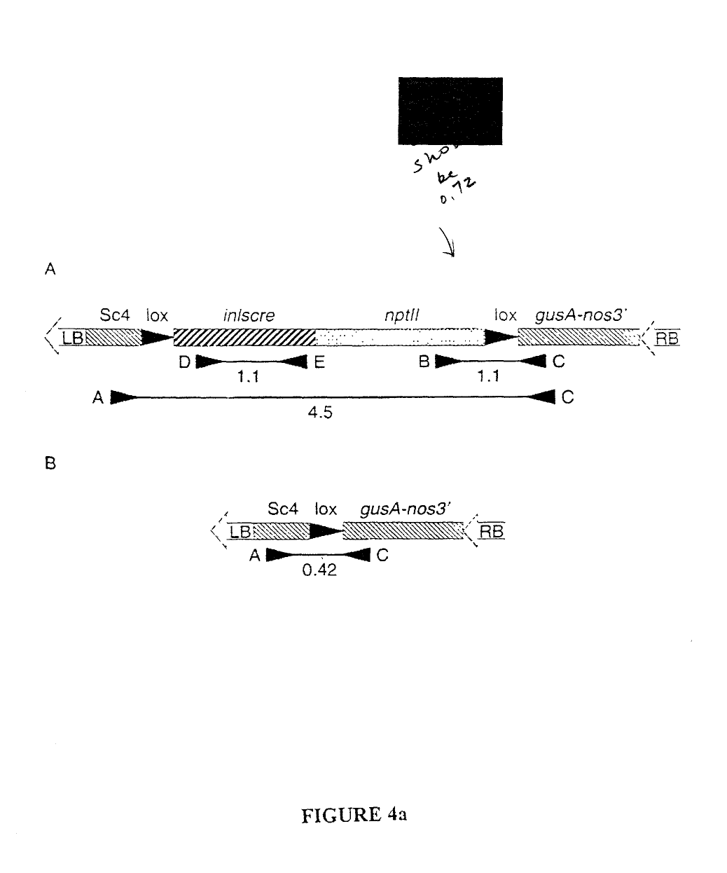

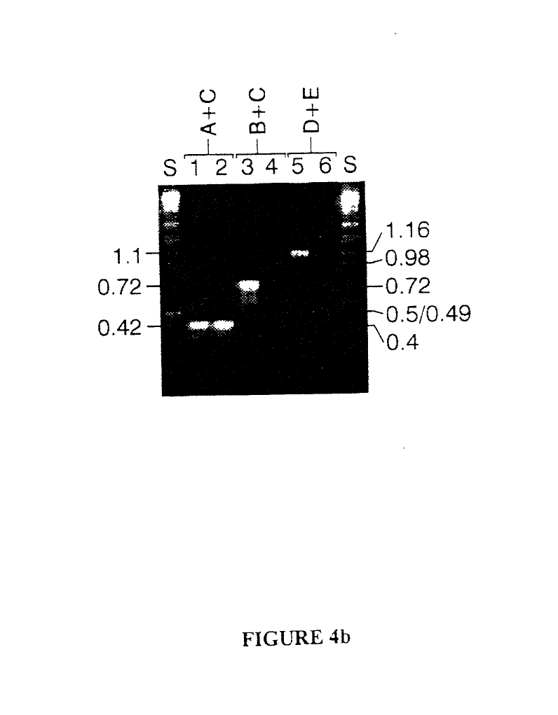

DNA was also extracted from the leaf tissue for PCR analysis, to determine whether excision had occurred. The rationale of this approach is outlined in FIG. 4a.

Using DNA obtained from ntBS229 plants prior to cre/lox-mediated recombination as template, PCR with primer combinations B+C and with D+E was calculated to produce amplification products of 0.72 kb and 1.1 kb in length, respectively (FIG. 4a, panel A). In contrast, no amplification products should be synthesised in PCR reactions using ntBS229 DNA isolated from plant material in which cre/lox-mediated recombination has occurred. This is because cre/lox-mediated excision of the nptII gene from genomic DNA prevents primer B from hybridising thereto (FIG. 4a, panel B).

Using DNA obtained from ntBS229 plants after cre/lox-mediated recombination has occurred as a template for PCR, the primer combination A+C was calculated to produce an amplification product of 0.42 kb in length (FIG. 4a, panel B). In contrast, the same primer pair was predicted to produce an amplification product of ˜4.5 kb in length, using DNA from ntBS229 plants in which no recombination has occurred (FIG. 4a, panel A).

As shown in FIG. 4b, amplification products of several ntBS229 T₀ leaf DNAs, of 0.72 kb, 1.1 kb and 0.42 kb in length, were obtained using the primer combinations B+C, D+E and A+C, respectively. These observations are consistent with the presence of both recombined and unrecombined pBS229 T-DNA constructs in the plant genomes.

In contrast, the ntBS229-4 regenerant which had significantly lower nptII activity contained only the excised construct, evident by the amplification of DNA of 0.42 kb in length only when primers A+C were used and no products when primers B+C or D+E were used (FIG. 4b).

Thus 9/10 leaves from five T₀ tobacco plants analysed were both GUS⁺, nptII⁺ and had a mixture of recombined and unrecombined pBS229 T-DNA constructs in their genomes. These plants were chimeric.

### EXAMPLE 8

### Excision of the nptII Gene from the Plant Genome of T₀ Regenerants

Plants were regenerated from leaf discs of one chimeric GUS⁺nptII⁺ T₀ tobacco plant, designated ntBS229-4. Thirteen plants, regenerated from six leaves, were assayed for both the GUS and nptII phenotype, and were subjected to PCR analysis of extracted DNA.

The regenerated plants were all GUS⁺ with expression evident in all tissues expected for Sc4 promoter-driven expression (data not shown).

PCR analysis of DNA extracted from these plants using primer combination A+C showed a product of 420 bp in all plants, while with primer combination B+C, a PCR product was seen only with DNA from plant #6, of 0.72 kb in length. The absence of any detectable level of cre/lox-mediated excision had increased in the ntBS229-4 regenerants compared to the parent ntBS229-4 plant. Furthermore, the cycle of tissue culture including regeneration employed was successful in reducing the frequency of chimeric plants produced.

NptII activity in 12/13 regenerated plants, was only slightly above background, however plant #6 had nptII activity levels characteristic of the chimeric parent ntBS229-4, from which it was derived (FIG. 3B). The background nptII activity levels in the 12 regenerants is indicative of residual nptII enzyme levels produced in cells prior to the excision of the nptII transcriptional unit from the genome.

To verify that cre/lox-mediated recombination had occurred in the regenerants, the 420 bp amplification product obtained from one of the regenerants using primers A+C was cloned and five independent clones subjected to DNA sequencing. The data (not shown) indicated that the expected cre/lox-mediated recombination event had indeed occurred.

Plants were similarly regenerated from three other GUS⁺nptII⁺T₀ tobaccos, ntBS229-8, -17 and -20. In comparison to plant ntBS229-4, where 12/13 regenerants were GUS^{+nptII}⁻, 4/18, 1/18 and 4/18 regenerants from ntBS229-8, -17 and -20 were GUS⁺nptII⁻, respectively.

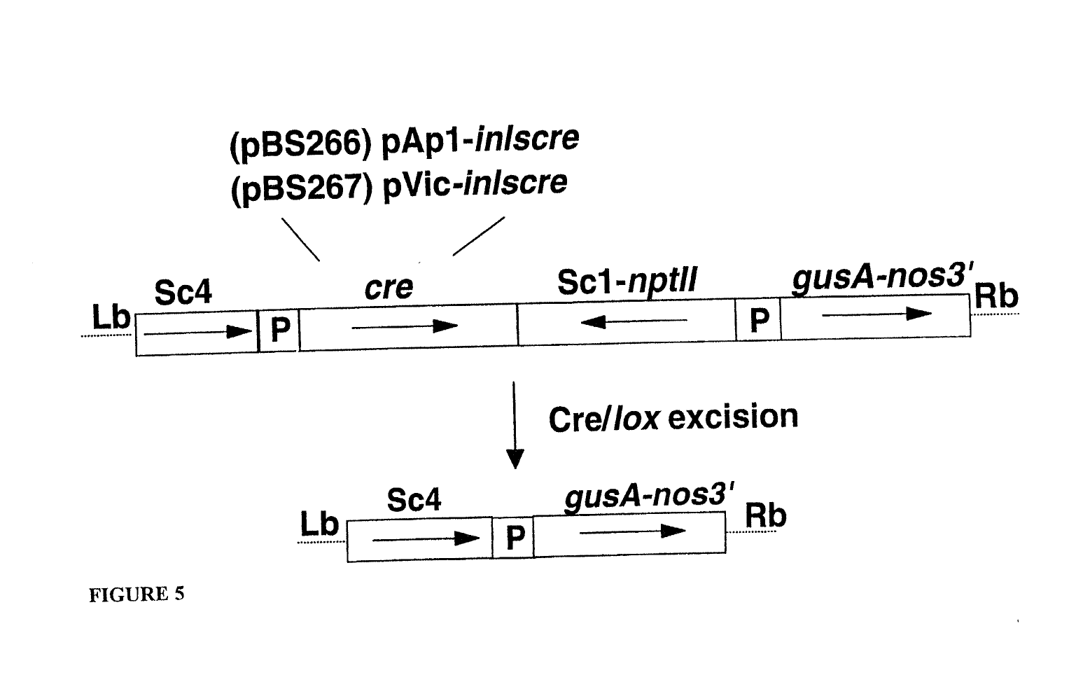

In a second experiment involving in planta cre/lox-mediated gene excision, the T-DNA regions of plasmids pBS229 (FIG. 1B), pBS266 and pBS267 (FIG. 5) were separately introduced into tobacco. The procedure used was as described above in Example 6 and 7, except that in this experiment transgenic tissue was regenerated in the light. T₀ tobacco plants were generated for each construct, and seed collected from these plants. Seeds were germinated, and T₁ seedlings analysed for GUS phenotype, nptII enzymatic activity and PCR analysis of extracted leaf DNA as described above in Example 7 and 8. The results of this analysis are shown in Table 2. It was found that three out of nine ntBS229 T₁ tobacco lines were GUS⁺nptII⁻, while with the nine ntBS266 and ntBS267 T₁ lines analysed, all 5 GUS⁺ lines in each case were also nptII⁺.

TABLE 2
GUS phenotype and nptII genotype of T₁ tobacco plants

| Source of T-DNA | T₁, GUS⁺nptII⁻ | T₁, GUS⁺nptII⁺ | T₁, GUS⁻nptII⁺ |
|---|---|---|---|
| pBS229 | 3/9^{a,b} | 1/9 | 5/9 |
| pBS266 | 0/9^{c} | 5/9 | 4/9 |
| pBS267 | 0/9 | 5/9 | 4/9 |

a: numbers in the table refer to the number of lines with the indicated phenotype and genotype, expressed as a proportion of the total number of T₁ lines analysed in each instance; the word “line” is used here to indicate lineage with the corresponding T₀ plant.

b: For each T₁ line, a minimum of 30 plants was scored for GUS phenotype by staining with X-gluc. To determine the NptII phenotype, nptII enzymatic assays were performed on at least 20 GUS⁺ T₁ plants for each construct; for each T₁ line, DNA from 2-3 GUS⁺ plants was then extracted and subjected to PCR analysis, to establish the nptII genotype.

c: PCR analysis of extracted DNA was not performed with ntBS266 T₁ tobacco lines.

In a third in planta cre/lox-mediated gene excision experiment, the T-DNA region of pBS229 was introduced into Solanum tuberosum cultivar Atlantic (potato) by Agrobacterium-mediated plant transformation (Peter Waterhouse, unpublished). 34 T₀ plants were regenerated and stained with X-gluc to determine the GUS phenotype. Two plants stained blue with X-gluc, indicating that cre/lox-mediated excision had occurred to produce a transcriptionally active gusA cassette (see FIG. 1). Plants are regenerated from tissue explants of the GUS⁺ stBS229 plants, and the regenerants characterised for GUS phenotype, nptII enzymatic assay and PCR analysis of extracted DNA as described above in Example 7 and 8.

### EXAMPLE 9

### Transformation in Planta Using a Hormone Gene for Selection of Transformed Tissue.

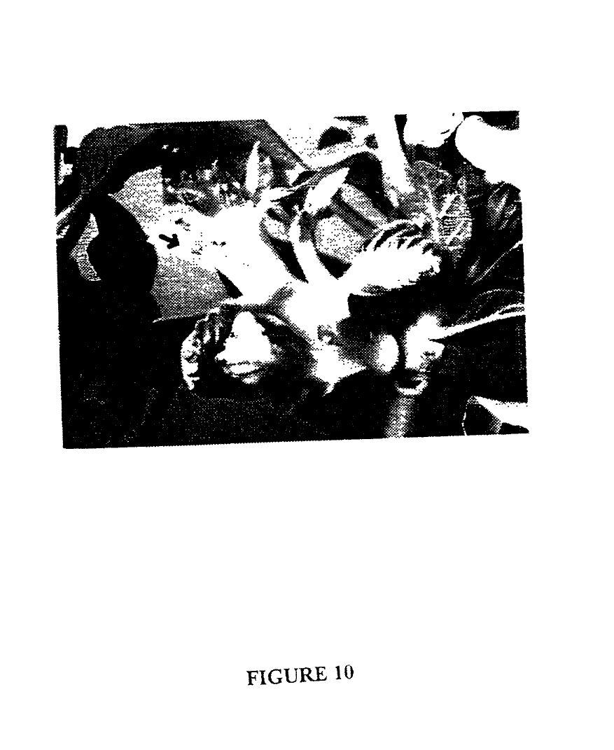

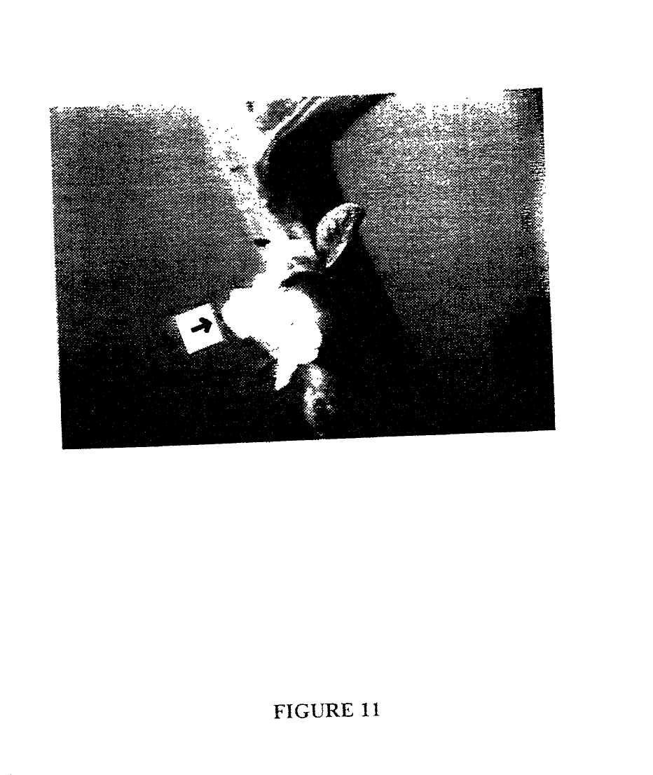

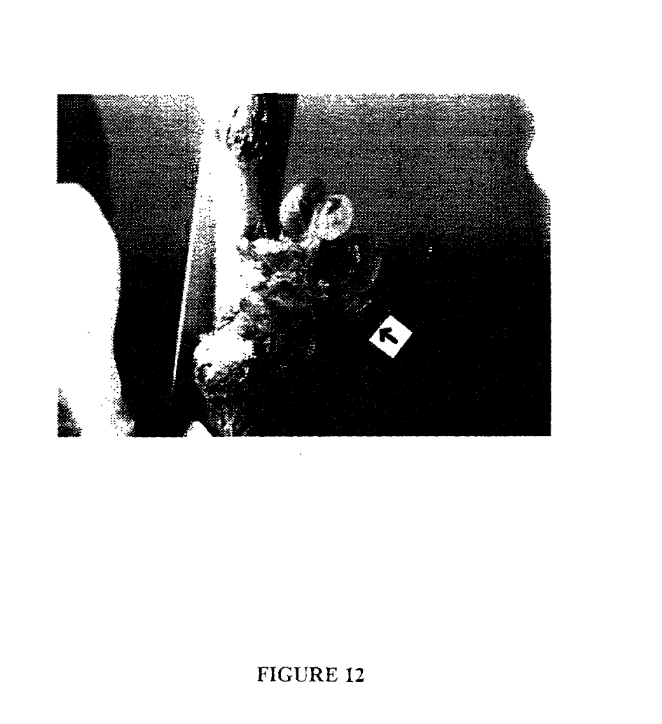

To demonstrate the principle of in planta selection of transformed tissue using a hormone gene, a construct was prepared which contained an ipt coding region and ipt 3′ polyadenylation sequence from the Agrobacterium tumefaciens pTiAch5 T-DNA (Heidekamp et al, 1983) inserted downstream of an enhanced 35S promoter and TMV 5′ untranslated leader region (Goelet et al, 1982) to confer strong constitutive in planta expression of isopentenyl transferase. In order to conduct Agrobacterium-mediated transformation of plant cells, the 35S-TMV5′-ipt-ipt3′-nos3′ gene from pRDF10072 was inserted into the binary vector pIG121-Hm (Hiei et al, 1994) to create pRDF10086 (FIG. 6). pRDF10086 and pIG121-Hm were separately introduced into Agrobacterium tumefaciens strain AGL1 (Lazo et al, 1991). Cultures of AGL1pRDF10086 and AGL1/pIG121-Hm were grown in the presence of 20 μM acetosyringone to induce vir gene expression, the cells harvested by centrifugation and concentrated 25-fold by resuspension of the cells in a small volume of sterile water. The bacterial suspensions were inoculated into stems of 6-week old tobacco plants (Nicotiana tabacum cv. Samsun NN) using a 23G needle attached to a syringe to puncture the stems. Plants were kept in the greenhouse at 23° C. daytime temperature for 3 days and then transferred to a 27° C. daytime/18° C. nighttime regime in the greenhouse. Galls appeared on plants 3 weeks after inoculation with AGL1/pRDF10086, after which time the plants were decapitated. No galls appeared on plants inoculated with AGL1/pIG121-Hm. Shoot primordia were visible on the surface of galls 5 weeks after inoculation and continued to develop and grow into shoots up to 10 cm long by 9 weeks after inoculation (FIG. 10,11,12). Many of the shoots were white to pale green in colour, had thickened stems and leaves, and showed loss of apical dominance, all typical symptoms of overexpression of cytokinin hormones in plant tissues. Some white or pale green shoots gave rise to leaves or parts of leaves that were (normal) green in colour.

### EXAMPLE 10

### Analysis of Tissues Arising After Agrobacterium-mediated Transfer of an ipt Gene

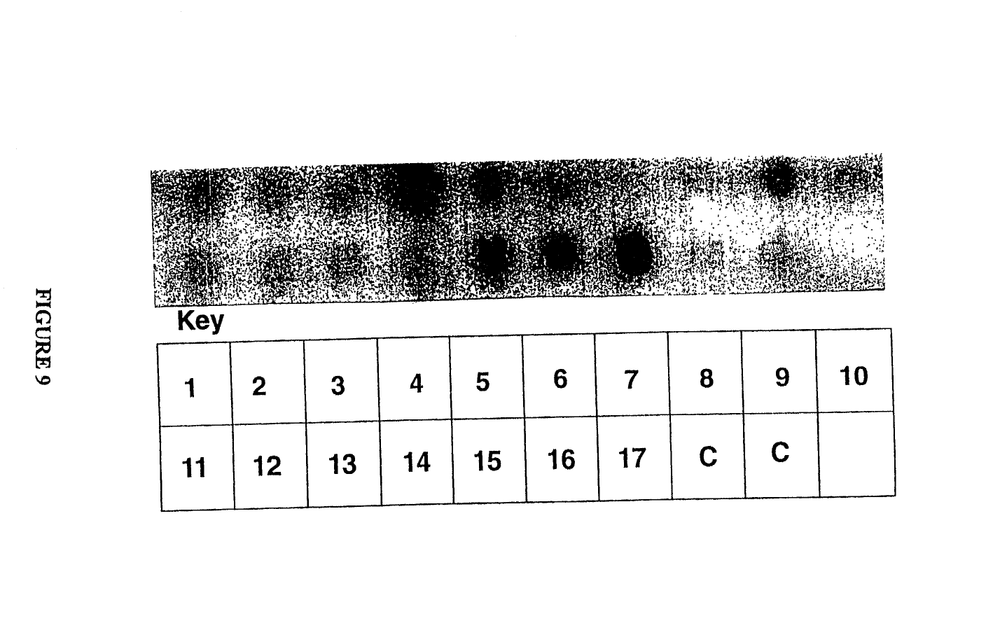

The T-DNA of pRDF10086 contains not only the ipt gene but also a nptII gene and a gusA gene (FIG. 6) driven by nos and 35S promoters, respectively, that can be used for detection of tansformed plant tissue by virtue of expression of neomycin phosphotransferase (Npt) and β-Glucuronidase (Gus) enzyme activities. Some galls, shoots and leaves that arose on tobacco plants inoculated with AGL1/pRDF10086 were analysed for Npt enzyme activity (McDonnell et al, 1987) and Gus activity by histochemical staining (Jefferson et al, 1987 ). Slices of gall tissue contained some Gus-positive zones in predominantly Gus-negative areas (data not shown). When shoots were analysed for Npt activity, 6/17 were Npt-positive (FIG. 9). Three of the Npt⁺ shoots were also Gus-positive. When leaves that were part green and part albino were stained for Gus activity, the albino areas were strongly Gus-positive while the green areas were Gus-negative, indicating inactivation of the gusA gene in the green zones, and suggesting that selection was operating against high level Gus and/or Ipt expression in some transformed tissues.

### EXAMPLE 11

### Description of Selection of Transformed Plant Tissue Using an Excisable Hormone Gene

Transformed shoots that are overexpressing the ipt gene are often phenotypically abnormal (eg see above) and are difficult to root (Smigocki and Owens, 1988). To obtain relatively normal tissues and whole plants from the ipt-transformed shoots, it is necessary to either inactivate or remove the ipt gene. One way this could be achieved is to use in planta inducible cre/lox-mediated gene excision in cis, with the ipt gene lying within the region of DNA bound by two lox sites, along with the inlscre gene. The genetic construct would normally contain a gene or genes, within the T-DNA but not within the region excised upon cre activation, for introduction into plant cells.

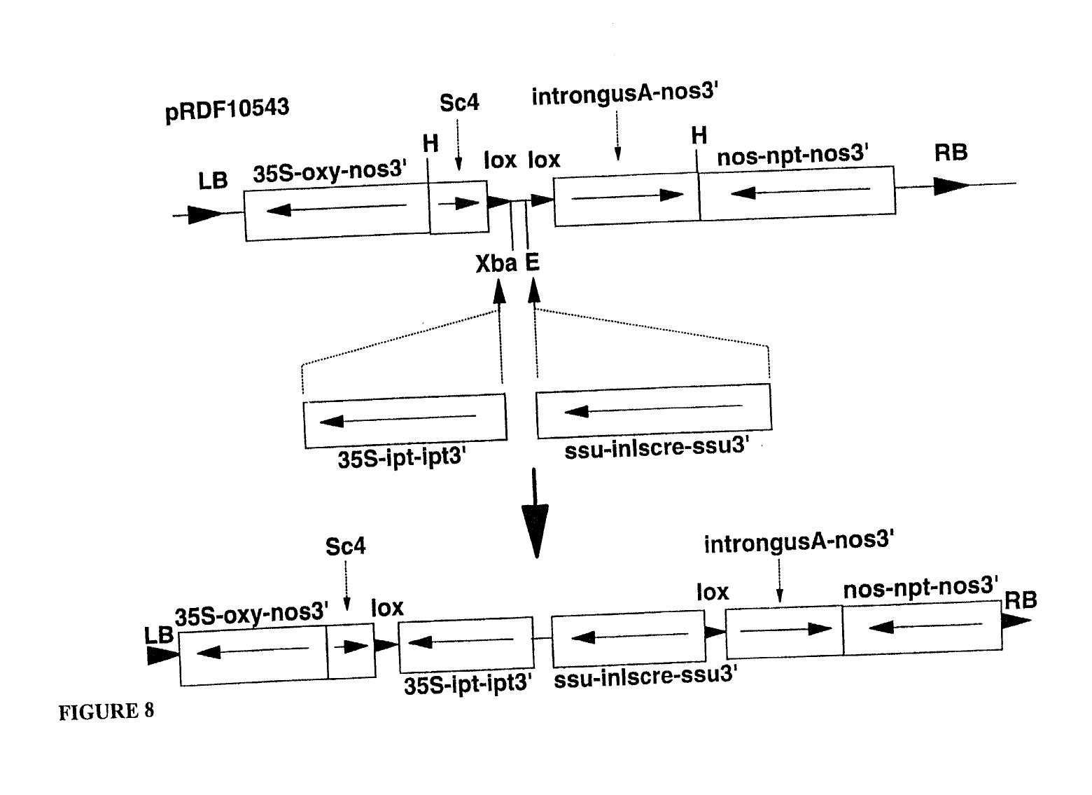

An example of such a genetic construct, presently under construction, is represented schematically in FIG. 8. A binary vector, pRDF10543, has been constructed as shown schematically in FIG. 7. This binary vector contains npt and oxy genes in addition to the Sc4-lox-lox-introngus-nos3′ cassette from pRDF10501. Two genes are inserted into pRDF10543, namely a 35S-ipt-ipt3′ gene from pRDF10072 and an ssu-inlscre-ssu3′ gene from prbcS-inlscre. Both are inserted between the lox recombination sites and are therefore be excised upon cre activation. The 35S-ipt gene functions in much the same way as demonstrated previously (Example 9) for the selection of transformed plant tissue. Sometime during or after formation of a shoot or other organised tissue resulting from Agrobacterium-mediated transfer of the genetic construct, expression of cre activity is induced, resulting in excision of the genes between the lox sites. The excisable cassette of the genetic construct is flanked by an Sc4 promoter on one side and a promoterless introngusA-nos3′ gene on the other side, such that inlscre-mediated excision of the excisable cassette results in juxtaposition of the Sc4 promoter to the gus gene, allowing expression of β-Glucuronidase enzyme. Activation of the gus gene is therefore an indicator of cre/lox-mediated excision. The genetic construct also contains nptII and oxy genes, conferring neomycin phosphotransferase (Npt) activity and resistance to the herbicide bromoxynil, respectively.

This genetic construct is introduced into Agrobacterium tumefaciens, and the resultant cells used to inoculate stems of tobacco plants as described earlier for AGL1/pRDF10086. Shoots and leaves that form from galls that grow at the inoculated sites are analysed for β-Glucuronidase and Npt enzyme activity and for survival after application of the herbicide bromoxynil (Rhône-Poulenc). Presence of either enzyme activity or resistance to bromoxynil indicates transformation of the plant tissues analysed. The presence of β-Glucuronidase enzyme activity indicates that excision of the excisable cassette has occurred in the transformed plant tissue. Excision of the ipt gene from such tissues results in a relatively normal phenotype of leaves and stems, namely greener leaves and stems with less thickening associated with overexpression of cytokinin hormones, compared to tissues retaining the ipt gene. Relatively normal looking, Gus-positive shoots are chosen for molecular analysis to demonstrate the presence of a reconstituted Sc4-gusA-nos3′ gene and to test for the presence and activity of the nptII and oxy genes. Shoots which show the presence of a reconstituted gus gene are allowed to flower and set seed, and progeny plants are analysed for segregation and activity of the nptII, gus and oxy genes. A Mendelian pattern of inheritance of one or more of these genes demonstrates that the chosen shoots were stably transformed by the genetic construct with subsequent excision of the ipt and inlscre genes.

Microorganism Deposits

The genetic constructs exemplified herein and designated pUC119-cre, pUC119-nlscre, pUC119-inlscre, p35S-cre, p35S-nlscre, p35S-inlscre, prbcS-inlscre, pBS210, pBS215, pBS229, pRDF10072, pRDF10086, pRDF10302, pRDF10453, pRDF10501, pRDF10278 and pRDF10543, have been deposited on Mar. 27, 1997 with the Australian Government Analytical Laboratories (AGAL), 1 Suakin Street, Pymble, New South Wales 2073, Australia, in accordance with and under the provisions of the Budapest Treaty on the International Recognition of the Deposit of Microorganisms for the Purposes of Patent Procedure, and assigned Accession Nos. ______, ______, ______, ______, ______, ______, ______, ______, ______, ______, ______, ______, ______, ______, ______, ______, and ______, respectively.

Equivalents

Those skilled in the art will appreciate that the invention described herein is susceptible to variations and modifications other than those specifically described. It is to be understood that the invention includes all such variations and modifications. The invention also includes all of the steps, features, compositions and compounds referred to in this specification, individually or collectively, and any and all combinations or any two or more of said steps or features.

References

1. Abremski et al (1983) Cell 32, 1301-1311

2. An et al (1985) EMBO J. 4:277-284.

3. Armstrong, C. L., Peterson, W. L., Buchholz, W. G., Bowen, B. A. Sulc, S. L. (1990). Plant Cell Reports 9, 335-339.

4. Boevink, P. et al (1995) Virology 207, 354-361

5. Christou, P., McCabe, D. E., Swain, W. F. (1988). Plant Physiol 87, 671-674.

6. Crossway et al (1986) Mol Gen. Genet. 202,179-185.

7. Dale, E. C., & Ow, D. W. (1991) Proc. Natl. Acad. Sci. 88, 10558-10562

8. de Feyter, R. et al (1997) Manuscript in preparation

9. Donald, R. G. K. & Cashmore, A. R. (1990) EMBO J. 9, 1717-1726

10. Ellis, J. G. et al (1987) EMBO J. 6, 11-16

11. Fromm et al (1985) Proc. Natl. Acad Sci. (USA) 82,5824-5828.

12. Goelet, P. et al (1982) Proc Natl Acad Sci USA 79, 5818-5822

13. Golic, K. G. et al (1989) Cell 59, 499-509

14. Graham, M. W. & Larkin, P. J. (1995) Transgenic Research 4, 324-331

15. Haseloff, J. and Gerlach, W. L. (1988). Nature 334, 586-594.

16. Heidekamp, F. et al (1983) Nucl Acids Res. 11, 6211-6223

17. Herrera-Estrella et al (1983a) Nature 303, 209-213.

18. Herrera-Estrella et al (1983b) EMBO J. 2, 987-995.

19. Herrera-Estrella et al (1985) In: Plant Genetic Engineering, Cambridge University Press, NY, pp 63-93.

20. Hiei, Y. et al (1994) Plant J. 6, 271-282

21. Jefferson, R. A. et al (1987) EMBO J. 6, 3901-3907

22. Kalderon, D. et al (1984) Cell 39, 499-509

23. Kay, R. et al (1987) Science 236, 1299-1302

24. Krens, F. A., Molendijk, L., Wullems, G. J. and Schilperoort, R. A. (1982). Nature 296, 72-74.

25. Landsmann, J. et al (1986) Nature 324, 166-168

26. Landsmann, J. et al (1989) Mol. Gen. Genet. 214, 68-73

27. Lazo, G. R. et al (1991) Bio/Technology 9, 963-967

28. Lloyd, A. M. & Davis, R. W. (1994) Mol. Gen. Genet. 242, 653-657

29. Lyznik, L. A. et al (1995) The Plant Journal 8, 177-186

30. Ma, Q-H et al (1997) Specific expression of isopentenyl transferase gene in transgenic tobacco seed controlled by a pea vicilin promoter Aust. J. Plant Physiol (submitted for publication)

31. Matzke, M. A. & Matzke A. J. M. (1995) Plant Physiol. 107, 679-68

32. McDonnell, R. E. et al (1987) Plant Mol. Biol. Rep. 5, 380-386

33. Murashige, T. & Skoog, F. (1962) Physiol. Plant. 15, 473-497

34. O'Gorman, S. et al (1991) Science 251, 1351-1355

35. Pazkowski et al (1984) EMBO J. 3, 2717-2722.

36. Russell, S. H. et al (1992) Mol. Gen. Genet. 234, 49-59

37. Sanger, F. et al (1977) Proc. Natl. Acad. Sci. 74, 5463-5467

38. Sambrook, J. et al (1989) In: Molecular Cloning—A laboratory manual (second edition), Cold Spring Harbor Lab. press

39. Smigocki, A. C. and Owens, L. D. (1988) Proc Natl Acad Sci USA 85, 5131-5135

40. Stalker, D. M. et al (1988) J. Biol Chem. 263, 6310-6314

41. Tabe, L. M. et al (1995) J. Anim. Sci. 73, 2752-2759

42. Vieira, J. & Messing, J. (1987) Methods Enzymol. 153, 3-11

1. A genetic construct comprising a first expression cassette which comprises:
(i) a recombinase genetic unit which comprises a genetic sequence which encodes a site-specific recombinase placed upstream of a terminator sequence and operably under the control of a first promoter; and
(ii) a transgene unit which comprises one or more expressable transgenes as hereinbefore deemed, placed operably under the control of one or more second promoter sequences;
wherein said recombinase genetic unit and said transgene unit are linked and wherein said first expression cassette is flanked by two recombination loci capable of binding to said site-specific recombinase.

2. The genetic construct according to claim 1 wherein the genetic sequence which encodes the site-specific recombinase is the cre gene and the recombination loci are lox sites or functionally-equivalent homologues, analogues or derivatives thereof.

3. The genetic construct according to claim 1 wherein the genetic sequence which encodes the site-specific recombinase is the flp gene and the recombination loci are frt sites or functionally-equivalent homologues, analogues or derivatives thereof.

4. The genetic construct according to any one of claims 1 to 3 wherein the transgene encodes a ribozyme molecule.

5. The genetic construct according to any one of claims 1 to 3 wherein the transgene encodes an antisense molecule.

6. The genetic construct according to any one of claims 1 to 3 wherein the transgene encodes a co-suppression molecule.

7. The genetic construct according to any one of claims 1 to 3 wherein the transgene is structural gene.

8. The genetic construct according to claim 7 wherein the structural gene sequence is a selectable marker gene, a reporter gene, a hormone gene, hormone-encoding gene, hormone-biosynthesis gene or a genetic sequence which encodes a polypeptide capable of regulating hormone levels.

9. The genetic construct according to claim 8 wherein the selectable marker gene is selected from the list comprising the antibiotic resistance genes which confer resistance to ampicillin, Claforan, gentamycin, G-418, hygromycin, kanamycin, neomycin, spectinomycin, tetracycline or a derivative or related compound thereto.

10. The genetic construct according to claim 8 wherein the selectable marker gene is selected from the list of herbicide-resistance genes which encode polypeptides which confer resistance to any one or more of atrazine, Basta, Bialophos, bromoxynil, Buctril, 2,4-D, glyphosate, phosphinothricin, sulphonylurea, or a derivative or related compound thereto.

11. The genetic construct according to claim 8 wherein the reporter gene is selected from the list comprising chloramphenicol acetyltransferase, β-glucuronidase, luciferase, and green fluorescent protein genes.

12. The genetic construct according to claim 8 wherein the structural gene encodes a polypeptide or enzyme which catalyses at least one step leading to the synthesis of a cytokinin or auxin or other plant growth regulator, or regulates the production or metabolism of said cytokinin, auxin or other plant growth regulator.

13. The genetic construct according to claim 12 wherein the structural gene is ipt.

14. The genetic construct according to any one of claims 1 to 13, wherein the genetic construct further comprises a modification to reduce or prevent recombinase expression in a prokaryote cell.

15. The geentic construct according to claim 14 wherein the modification is the insertion of an intron sequence to disrupt expression of the recombinase genetic unit absent removal of said intron sequence.

16. The genetic construct according to claim 14 wherein the modification is the insertion of an intron sequence in the coding region of the recombinase gene.

17. The genetic construct according to any one of claims 1 to 16 wherein the first and second promoters are capable of conferring expression of the structural gene and site-specific recombinase gene in a eukaryote cell.

18. The genetic construct according to claim 17 wherein the eukaryote is a plant.

19. The genetic construct according to claim 18 wherein the plant is selected from the list comprising stolon-bearing or tuber-bearing plants such as potatoes, sweet potatoes, jerusalem artichoke, taro or yams, fibre or wood tree crops such as Eucalyptus ssp. or Pinus ssp., aspen, ornamental plants such as gerberas, chrysanthemum, orchids, lilies, roses, fuschias, azaleas carnations, camellias or gardenias, citrus crops such as oranges, lemons, grapefruit, tangerines or limes, fruit tress such as apples or pears, berry fruits such as strawberry, raspberry, loganberry or blackberry, tropical crops such as sugarcane, tobacco, bananas, plantain or pineapples or asparagus.

20. The genetic construct according to claim 18 wherein the plant is a solanaceous plant.

21. The genetic construct according to claim 20 wherein the plant is tobacco or potato.

22. The genetic construct according to any one of claims 1 to 17 wherein the first and/or second promoter is selected from the list comprising constitutive promoters, seed-specific promoters, floral-specific promoters, anther-specific promoters, tapetum-specific promoters, root-specific promoters, leaf-specific promoters, stem-specific promoters, meristem-specific promoters, light-inducible promoters, metal-inducible promoters, heat-shock promoters, wound-inducible and stress-inducible promoters.

23. The genetic construct according to claim 22 wherein the first and/or second promoters are selected from the list comprising CaMV 35S, NOS, OCS, Sc1, Sc4 and rbcS, amongst other.

24. The genetic construct according to claims 22 or 23 wherein the first promoter is an inducible promoter.

25. The genetic construct according to claim 24 wherein the inducible promoter is the rbcS promoter.

26. The genetic construct according to claim 25 wherein the first promoter is the Arabidopsis thaliana rbcS 1a promoter.

27. The genetic construct according to claim 23 wherein the first promoter is the CaMV 35S promoter.

28. The genetic construct according to any one of claims 23 to 27 wherein the second promoter is the Sc4 promoter.

29. The genetic construct according to any one of claims 1 to 28 wherein the first promoter switches on expression of the site-specific recombinase following the commencement of expression of the structural gene sequence.

30. The genetic construct according to claim 29 wherein the first promoter is the Arabidopsis thaliana rbcS 1a promoter and the second promoter is the CaMV 35S promoter or the Sc4 promoter.

31. The genetic construct according to any one of claims 1 to 30 wherein the recombinase genetic unit further comprises a nucleotide sequence which encodes a nuclear localisation signal fused in-frame to the coding region of the recombinase gene.

32. The genetic construct according to claim 31 wherein the nuclear localisation signal is the SV40 T-antigen type nuclear localisation signal.

33. The genetic construct according to any one of claims 1 to 32 wherein the first expression cassette flanked by recombination loci is inserted into a second expression cassette such that excision of the first expression cassette from the second expression cassette alters expression of the second expression cassette.

34. The genetic construct according to claim 33 wherein the second expression cassette comprises one or more expressable transgenes selected from the list comprising structural genes, ribozymes, antisense molecules or co-suppression molecules and wherein each of said transgenes is placed operably under the control of a promoter sequence.

35. The genetic construct according to claim 34 wherein the transgene of the second expression cassette is a structural gene.

36. The genetic construct according to claim 35 wherein the structural gene is a reporter gene.

37. The genetic construct according to any one of claims 33 to 36 wherein the transgene of the second expression cassette is expressed following excision of the first expression cassette.

38. The genetic construct according to any one of claims 1 to 32 further comprising an expressable transgene operably connected to a promoter sequence wherein said expressable transgene is juxtaposed to the outside of the region flanked by the recombination loci and separated from the adjacent recombination loci by a spacer region of at least 2 nucleotides in length.

39. The genetic construct according to claim 38 wherein the expressible gene encodes a functional enzyme, polypeptide, ribozyme, antisense, co-suppression molecule or other RNA molecule.

40. The genetic construct according to any one of claims 1 to 39 further comprising one or more left border and/or right border sequences or other T-DNA sequences to facilitate its in vivo insertion into plant chromosomal DNA.

41. The genetic construct according to any one of claims 1 to 40 when used to transform a cell.

42. The genetic construct according to any one of claims 1 to 40 when used to delete, excise or otherwise remove a transgene from a transformed cell.

43. A method of removing a transgene from a cell transformed with the genetic construct according to any one of claims 1 to 40, said method comprising expressing the recombinase genetic unit of said genetic construct for a time and under conditions sufficient for a site-specific recombinase to be expressed and at least excise the first expression cassette of said genetic construct.

44. A method of transiently expressing a transgene in a stably transformed cell, said method comprising:
(i) stably transforming said cell with the genetic construct according to any one of claims 1 to 40;
(ii) expressing the Transgene of the transgene unit in said stably transformed cell; and
(iii) expressing the recombinase genetic unit of said genetic construct for a time and under conditions sufficient for a site-specific recombinase to be expressed and at least excise the first expression cassette of said genetic construct.

45. The method according to claims 43 or 44 wherein the transgene is selected from the list comprising structural genes, ribozymes, antisense molecule and co-suppression molecules.

46. The method according to claim 45 wherein the expressible transgene is a structural gene selected from the list comprising selectable marker gene, reporter gene, hormone gene, hormone-encoding gene, hormone biosynthesis gene or genetic sequence which encodes a polypeptide capable of regulating hormone levels.

47. A method of inducing, suppressing or otherwise altering the expression of a transgene in a cell transformed with the genetic construct according to claim 33, said method comprising expressing the recombinase genetic unit of said genetic construct for a time and under conditions sufficient for a site-specific recombinase to be expressed and at least excise the first expression cassette of said genetic construct.

48. A method of producing a transformed cell comprising the steps of:
(i) transforming a cell with the genetic construct according to any one of claims 1 to 40; and
(ii) expressing the recombinase genetic unit for a time and under conditions sufficient for expression of the site-specific recombinase encoded by said recombinase genetic unit to occur and result in excision of the transgene of the first expression cassette of said genetic construct or a fragment thereof sufficient to disrupt expression of said transgene.

49. The method according to claim 48 wherein the transgene of the first expression cassette comprises a selectable marker gene and the step of expressing the recombinase genetic unit results in excision of said selectable marker gene or a fragment thereof sufficient to prevent its expression.

50. The method according to claim 49 wherein the selectable marker gene is selected from the list comprising the antibiotic resistance genes which confer resistance to ampicillin, Claforan, gentamycin, GA-418, hygromycin, kanamycin, neomycin, spectinomycin, tetracycline or a derivative or related compound thereto.

51. The method according to claim 40 wherein the selectable marker gene is selected from the list of herbicide-resistance genes which encode polypeptides which confer resistance to any one or more of atrazine, Basta, Bialophos, bromoxynil, Buctril, 2,4-D, glyphosate, phosphinothricin, sulphonylurea, or a derivative or related compound thereto.

52. A method of producing a transformed plant cell, said method comprising the steps of:
(i) transforming said cell with the genetic construct according to any one of claims 12 to 40, wherein the structural gene of the first expression cassette is a hormone gene, hormone-encoding gene, hormone-biosynthesis gene or genetic sequence which encodes a polypeptide capable of regulating hormone levels;
(ii) expressing said structural gene in said transformed cell for a time and under conditions sufficient for said cell to differentiate into the progenitor cells of said organ;
(iii) expressing the recombinase genetic unit of the genetic construct for a time and under conditions sufficient for expression of the site-specific recombinase encoded by said recombinase genetic unit to occur, thereby leading to excision of the structural gene of the first expression cassette or a fragment thereof sufficient to disrupt expression of the structural gene.

53. The method according to claim 52 comprising the additional step of growing the differentiated progenitor cell into an organ or whole plant.

54. The method according to claim 52 or 53 wherein the cell which is transformed in step (i) is derived from a plant cell line, suspension culture of a plant cell line, tissue culture of a plant cell, or callus.

55. The method according to claim 52 or 53 wherein the cell which is transformed in step (i) is derived from a tissue explant selected from the list comprising leaf, stem, root, or seed, amongst others.

56. The method according to claim 52 or 53 wherein the transformation step (i) is carried out in situ on a whole plant.

57. The method according to any one of claims 52 to 56 wherein the hormone gene, hormone-encoding gene, hormone-biosynthesis gene or genetic sequence which encodes a polypeptide capable of regulating hormone levels produces a cytokinin or regulates the production or metabolism of a cytokinin when expressed in the plant cell, sufficient to result in adventitious shoot formation.

58. The method according to claim 57 wherein the hormone gene, hormone-encoding gene, hormone-biosynthesis gene or genetic sequence which encodes a polypeptide capable of regulating hormone levels is ipt or a homologue, analogue or derivative thereof.

59. The method according to any one of claims 52 to 56 wherein the hormone gene, hormone-encoding gene, hormone-biosynthesis gene or genetic sequence which encodes a polypeptide capable of regulating hormone levels produces an auxin or regulates the production or metabolism of an auxin when expressed in the plant cell, sufficient to result in adventitious root formation.

60. The method according to any one of claims 52 to 56 wherein the hormone gene, hormone-encoding gene, hormone-biosynthesis gene or genetic sequence which encodes a polypeptide capable of regulating hormone levels produces a gibberellin or regulates the production or metabolism of an gibberellin when expressed in the plant cell, sufficient to result in organogenesis.

61. A method of introducing multiple genes into a cell using a single selectable marker gene, said method comprising the steps of:
(i) transforming said cell with a genetic construct, according to any one of claims 33 to 40 wherein transgene of the first expression cassette is a selectable marker gene;
(ii) expressing the recombinase gene contained in said genetic construct in said cell or the progeny of said cell; and
(iii) transforming the cell obtained in step (ii) with a second genetic construct as hereinbefore described, wherein the structural gene of said genetic construct is a selectable marker gene which is substantially the same as the selectable marker gene use in step (i) or a homologue, analogue or a derivative thereof.

62. The method according to claim 61 comprising the further step of repeating step (ii) of said method.

63. The method according to claim 62 further comprising repeating the steps defined by claim 61 at least once.

64. A cell or organism transformed with the genetic construct according to any one of claims 1 to 40 or a derivative thereof produced by the removal of the first expression cassette of said genetic construct therefrom.

65. The cell or organism according to claim 64 further characterised as a prokaryotic cell.

66. The cell or organism according to claim 64 further characterised as a eukaryotic cell or organism.

67. The cell or organism according to claim 64 wherein the eukaryote cell is a plant cell or organism.

68. A cell or organism which comprises a footprint of at least about 30 nucleotides in length derived from the genetic construct according to any one of claims 1 to 40, wherein said footprint at least comprises one the recombination loci of said genetic construct.

69. The cell or organism according to claim 68 wherein the plant is selected from the list comprising stolon-bearing or tuber-bearing plants such as potatoes, sweet potatoes, jerusalem artichoke, taro or yams, fibre or wood tree crops such as Eucalyptus ssp. or Pinus ssp., aspen, ornamental plants such as gerberas, chrysanthemum, orchids, lilies, roses, fuschias, azaleas carnations, camellias or gardenias, citrus crops such as oranges, lemons, grapefruit, tangerines or limes, fruit tress such as apples or pears, berry fruits such as strawberry, raspberry, loganberry or blackberry, tropical crops such as sugarcane, tobacco, bananas, plantain or pineapples or asparagus, amongst others.

70. The genetic construct according to any one of claims 1 to 40 when used to ablate a cell or tissue in planta.
